(19) United States
(12) Patent Application Publication
(10) Pub. No.: US 2024/0317776 A1
(43) Pub. Date: Sep. 26, 2024

(54) SOLID FORMS OF 4-(3,3-DIFLUORO-2,2-DIMETHYL-PROPANOYL)-3,5-DIHYDRO-2H-PYRIDO[3,4-F][1,4]OXAZEPINE-9-CARBONITRILE

(71) Applicant: Denali Therapeutics Inc., South San Francisco, CA (US)

(72) Inventors: Javier de Vicente Fidalgo, South San Francisco, CA (US); Brian M. Fox, South San Francisco, CA (US); Christopher R.H. Hale, South San Francisco, CA (US)

(73) Assignee: Denali Therapeutics Inc., South San Francisco, CA (US)

(21) Appl. No.: 18/582,522

(22) Filed: Feb. 20, 2024

Related U.S. Application Data
(63) Continuation of application No. 18/225,236, filed on Jul. 24, 2023, now abandoned.
(60) Provisional application No. 63/259,921, filed on Jul. 25, 2022.

(51) Int. Cl. C07D 498/04

(52) U.S. Cl. CPC C07D 498/04; C07B 2200/13

(57) ABSTRACT

Described herein are solid forms of 4-(3,3-difluoro-2,2-dimethyl-propanoyl)-3,5-dihydro-2H-pyrido[3,4-f][1,4]oxazepine-9-carbonitrile, the process of preparing the forms, pharmaceutical compositions comprising same, and methods of use thereof.

### CROSS-REFERENCE TO RELATED APPLICATIONS

This application is a continuation of U.S. application Ser. No. 18/225,236, filed Jul. 24, 2023, which claims priority to U.S. Provisional Application No. 63/259,921, filed on Jul. 25, 2022, which are incorporated by reference herein in their entirety for any purpose.

### BRIEF SUMMARY OF THE DISCLOSURE

Described herein are solid forms of 4-(3,3-difluoro-2,2-dimethyl-propanoyl)-3,5-dihydro-2H-pyrido[3,4-f][1,4]oxazepine-9-carbonitrile, the process of preparing the forms, pharmaceutical compositions, and methods of use thereof.

### BACKGROUND OF THE INVENTION

Receptor-interacting protein kinase 1 (RIPK1) is a key regulator of inflammation, apoptosis, and necroptosis. RIPK1 has an important role in modulating inflammatory responses mediated by nuclear-factor kappa-light chain enhancer of activated B cells (NF-kB). More recent research has shown that its kinase activity controls necroptosis, a form of necrotic cell death. Further, RIPK1 is part of a pro-apoptotic complex indicating its activity in regulating apoptosis. Dysregulation of receptor-interacting protein kinase 1 signaling can lead to excessive inflammation or cell death. Research suggests that inhibition of RIPK1 is a potential clinical target for diseases involving inflammation or cell death. RIPK1 kinase has emerged as a promising therapeutic target for the treatment of a wide range of human neurodegenerative, autoimmune, and inflammatory diseases.

The compound of Formula (1), 4-(3,3-difluoro-2,2-dimethyl-propanoyl)-3,5-dihydro-2H-pyrido[3,4-f][1,4]oxazepine-9-carbonitrile, (hereinafter also referred as “Compound (1)”), depicted below, is a RIPK1 inhibitor:

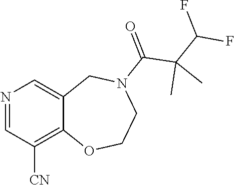 [SMILES: CC(C)(C(=O)N1CCOc2c(C#N)cncc2C1)C(F)F]

One factor in assessing the suitability of a compound as a therapeutic agent is whether the compound as a therapeutic agent can be administered in a form that is easily absorbed by the body and also shelf-stable. The pharmaceutically active substance used to prepare the treatment should be as pure as possible and its stability on long-term storage should be guaranteed under various environmental conditions. These properties are useful to prevent the appearance of unintended degradation products in pharmaceutical compositions, which degradation products may be potentially toxic or result simply in reducing the potency and/or efficacy of the composition.

A primary concern for the large-scale manufacture of pharmaceutical compounds is that the active substance should have a stable crystalline morphology to ensure consistent processing parameters and pharmaceutical quality. If an unstable crystalline form is used, crystal morphology may change during manufacture and/or storage, resulting in quality control problems and formulation irregularities. Such a change may affect the reproducibility of the manufacturing process and thus lead to final formulations which do not meet the high quality and stringent requirements imposed on formulations of pharmaceutical compositions. In this regard, it should be generally borne in mind that any change to the solid state of a pharmaceutical composition which can improve its physical and chemical stability gives a significant advantage over less stable forms of the same drug.

When a compound crystallizes from a solution or slurry, it may crystallize with different spatial lattice arrangements, a property referred to as “polymorphism.” Each of the crystal forms is a “polymorph.” Although polymorphs of a given substance have the same chemical composition, they may differ from each other with respect to one or more physical properties, such as solubility, dissociation, true density, dissolution, melting point, crystal shape, morphology, particle size, compaction behavior, flow properties, and/or solid-state stability.

Although it is known that the preparation of crystalline forms may improve the physical or pharmaceutical properties of a pharmaceutically active compound, it is not possible to predict whether a compound exists in crystalline form(s) or which crystalline form(s) may possess advantages for a particular purpose prior to the actual preparation and characterization of the crystalline form. In particular, such advantages, in a non-limiting manner could include better processability, solubility or shelf-life stability, just to name a few. Other advantages may also include biological properties such as improved bioavailability, reduced adverse reactions at the GI tract (for example irritation of the GI tract, partial degradation of the compound, etc.), or better deliverability of the drug to the intended target site among other advantages.

### SUMMARY OF THE DISCLOSURE

The present disclosure relates to various solid state forms of the RIPK 1 inhibitor 4-(3,3-difluoro-2,2-dimethyl-propanoyl)-3,5-dihydro-2H-pyrido[3,4-f][1,4]oxazepine-9-carbonitrile (i.e., Compound (1)), the process of preparing the forms, and pharmaceutical compositions and methods of use thereof.

Disclosed herein is a solid form of 4-(3,3-difluoro-2,2-dimethyl-propanoyl)-3,5-dihydro-2H-pyrido[3,4-f][1,4]oxazepine-9-carbonitrile that is characterized as crystalline Form A.

Also disclosed herein is an amorphous form of 4-(3,3-difluoro-2,2-dimethyl-propanoyl)-3,5-dihydro-2H-pyrido[3,4-f][1,4]oxazepine-9-carbonitrile.

Also disclosed herein is pharmaceutical composition comprising the solid forms of 4-(3,3-difluoro-2,2-dimethyl-propanoyl)-3,5-dihydro-2H-pyrido[3,4-f][1,4]oxazepine-9-carbonitrile disclosed herein and a pharmaceutically acceptable carrier.

Still further disclosed herein is a method of treating a disease and condition mediated by RIPK 1 in a patient in need thereof, comprising administering to the patient an effective amount of the solid forms of 4-(3,3-difluoro-2,2-dimethyl-propanoyl)-3,5-dihydro-2H-pyrido[3,4-f][1,4]oxazepine-9-carbonitrile disclosed herein.

The present disclosure also relates to the solid forms of 4-(3,3-difluoro-2,2-dimethyl-propanoyl)-3,5-dihydro-2H-pyrido[3,4-f][1,4]oxazepine-9-carbonitrile disclosed herein for use in treating a disease and condition mediated by RIPK1 in a patient in need thereof.

The present disclosure further relates to use of the disclosed solid forms of 4-(3,3-difluoro-2,2-dimethyl-propanoyl)-3,5-dihydro-2H-pyrido[3,4-f][1,4]oxazepine-9-carbonitrile in the manufacture of a medicament for treating a disease involving mediation of the RIPK1 receptor.

### BRIEF DESCRIPTION OF THE DRAWINGS

FIG. 1 shows an X-ray powder diffractogram of crystalline Form A of 4-(3,3-difluoro-2,2-dimethyl-propanoyl)-3,5-dihydro-2H-pyrido[3,4-f][1,4]oxazepine-9-carbonitrile.

FIG. 2 shows a Differential Scanning Calorimetry/Thermal Gravimetric Analysis (DSC/TGA) thermogram of crystalline Form A of 4-(3,3-difluoro-2,2-dimethyl-propanoyl)-3,5-dihydro-2H-pyrido[3,4-f][1,4]oxazepine-9-carbonitrile.

FIG. 3 shows a polarized light microscopy image of crystalline Form A of 4-(3,3-difluoro-2,2-dimethyl-propanoyl)-3,5-dihydro-2H-pyrido[3,4-f][1,4]oxazepine-9-carbonitrile.

FIG. 4 shows a dynamic vapor sorption isotherm plot of crystalline Form A of 4-(3,3-difluoro-2,2-dimethyl-propanoyl)-3,5-dihydro-2H-pyrido[3,4-f][1,4]oxazepine-9-carbonitrile.

FIG. 5 shows an overlay of X-ray powder diffractograms of crystalline Form A of 4-(3,3-difluoro-2,2-dimethyl-propanoyl)-3,5-dihydro-2H-pyrido[3,4-f][1,4]oxazepine-9-carbonitrile before and after dynamic vapor sorption.

FIG. 6 shows an HPLC chromatogram of crystalline Form A of 4-(3,3-difluoro-2,2-dimethyl-propanoyl)-3,5-dihydro-2H-pyrido[3,4-f][1,4]oxazepine-9-carbonitrile.

FIG. 7 provides Yasuda-Shedlovsky plots of pKa measurement for crystalline Form A of 4-(3,3-difluoro-2,2-dimethyl-propanoyl)-3,5-dihydro-2H-pyrido[3,4-f][1,4]oxazepine-9-carbonitrile.

FIG. 8 shows a polarized light microscopy image of a single crystal of crystalline Form A of 4-(3,3-difluoro-2,2-dimethyl-propanoyl)-3,5-dihydro-2H-pyrido[3,4-f][1,4]oxazepine-9-carbonitrile.

FIG. 9 shows an asymmetrical unit representation of crystalline Form A of 4-(3,3-difluoro-2,2-dimethyl-propanoyl)-3,5-dihydro-2H-pyrido[3,4-f][1,4]oxazepine-9-carbonitrile.

FIG. 10 shows a thermal ellipsoid (ORTEP) representation of crystalline Form A of 4-(3,3-difluoro-2,2-dimethyl-propanoyl)-3,5-dihydro-2H-pyrido[3,4-f][1,4]oxazepine-9-carbonitrile.

FIG. 11 shows the predicted chemical structure of Compound (1) as determined by single crystal analysis.

FIG. 12 shows a unit cell of crystalline Form A of 4-(3,3-difluoro-2,2-dimethyl-propanoyl)-3,5-dihydro-2H-pyrido[3,4-f][1,4]oxazepine-9-carbonitrile.

FIG. 13 shows a packing diagram of crystalline Form A of 4-(3,3-difluoro-2,2-dimethyl-propanoyl)-3,5-dihydro-2H-pyrido[3,4-f][1,4]oxazepine-9-carbonitrile shown along the a-axis.

FIG. 14 shows a packing diagram of crystalline Form A of 4-(3,3-difluoro-2,2-dimethyl-propanoyl)-3,5-dihydro-2H-pyrido[3,4-f][1,4]oxazepine-9-carbonitrile shown along the b-axis.

FIG. 15 shows a packing diagram of crystalline Form A of 4-(3,3-difluoro-2,2-dimethyl-propanoyl)-3,5-dihydro-2H-pyrido[3,4-f][1,4]oxazepine-9-carbonitrile shown along the c-axis.

FIG. 16 shows an overlay of X-ray powder diffractograms comparing crystalline Form A of 4-(3,3-difluoro-2,2-dimethyl-propanoyl)-3,5-dihydro-2H-pyrido[3,4-f][1,4]oxazepine-9-carbonitrile starting material, experimental single crystal, and calculated single crystal.

FIG. 17 shows an overlay of X-ray powder diffractograms comparing crystalline Form A of 4-(3,3-difluoro-2,2-dimethyl-propanoyl)-3,5-dihydro-2H-pyrido[3,4-f][1,4]oxazepine-9-carbonitrile starting material, and after 1 week of storage under the following conditions: 40° C./75% RH, 25° C./60% RH, 60° C.

FIG. 18 shows an overlay of X-ray powder diffractograms comparing crystalline Form A of 4-(3,3-difluoro-2,2-dimethyl-propanoyl)-3,5-dihydro-2H-pyrido[3,4-f][1,4]oxazepine-9-carbonitrile starting material, and after 4 weeks of storage under the following conditions: 40° C./75% RH, 25° C./60% RH, 60° C.

FIG. 19 shows the kinetic solubility curves of crystalline Form A of 4-(3,3-difluoro-2,2-dimethyl-propanoyl)-3,5-dihydro-2H-pyrido[3,4-f][1,4] oxazepine-9-carbonitrile in various biorelevant media at 37° C.

FIG. 20 shows an overlay of X-ray powder diffractograms comparing crystalline Form A of 4-(3,3-difluoro-2,2-dimethyl-propanoyl)-3,5-dihydro-2H-pyrido[3,4-f][1,4] oxazepine-9-carbonitrile before and after various solubility tests at 37° C.

FIG. 21 shows an overlay of X-ray powder diffractograms comparing crystalline Form A of 4-(3,3-difluoro-2,2-dimethyl-propanoyl)-3,5-dihydro-2H-pyrido[3,4-f][1,4]oxazepine-9-carbonitrile before and after various solubility tests at RT.

FIG. 22 shows an overlay of X-ray powder diffractograms comparing crystalline Form A of 4-(3,3-difluoro-2,2-dimethyl-propanoyl)-3,5-dihydro-2H-pyrido[3,4-f][1,4] oxazepine-9-carbonitrile before and after various pH solubility tests.

FIG. 23 shows an LC chromatogram and mass spectra of crystalline Form A of 4-(3,3-difluoro-2,2-dimethyl-propanoyl)-3,5-dihydro-2H-pyrido[3,4-f][1,4]oxazepine-9-carbonitrile after 24 hrs in pH 2.0.

FIG. 24 shows an LC chromatogram and mass spectra of crystalline Form A of 4-(3,3-difluoro-2,2-dimethyl-propanoyl)-3,5-dihydro-2H-pyrido[3,4-f][1,4]oxazepine-9-carbonitrile after 24 hrs in pH 8.0.

FIG. 25 shows an LC chromatogram and mass spectra of crystalline Form A of 4-(3,3-difluoro-2,2-dimethyl-propanoyl)-3,5-dihydro-2H-pyrido[3,4-f][1,4]oxazepine-9-carbonitrile after 96 hrs in pH 8.0.

FIG. 26 shows the free energy landscape at 298.15 K from step 4 of the calculations as discussed in Example 5.

FIG. 27 shows an overlay of the molecular conformations in rank 1 (middle structure), rank 5 (top structure), and rank 6 (lower structure), with hydrogen atoms omitted for clarity. The diagram shows the molecular flexibility of-(3,3-difluoro-2,2-dimethyl-propanoyl)-3,5-dihydro-2H-pyrido[3,4-f][1,4]oxazepine-9-carbonitrile.

FIG. 28 shows a similarity matrix of the 30 most stable predicted structures, with values from 0.8 to 1.0 highlighted on a white-grey color scale.

FIG. 29 shows an overlay of the molecular conformations of rank 1 (white), rank 2 (crosshatch), and rank 3 (black). The structures only overlay in projection, not in three dimensions.

FIG. 30 shows an overlay of the single crystal structure of Form A (white) with rank 1 (black).

FIG. 31 shows the free energy landscape with the experimental forms indicated.

FIG. 32 shows the free energy landscape as a function of temperature.

FIG. 33 shows the XRPD spectrum of an amorphous form of 4-(3,3-difluoro-2,2-dimethyl-propanoyl)-3,5-dihydro-2H-pyrido[3,4-f][1,4]oxazepine-9-carbonitrile as described hereinbelow.

FIG. 34 shows a Differential Scanning Calorimetry (DSC) thermogram of amorphous 4-(3,3-difluoro-2,2-dimethyl-propanoyl)-3,5-dihydro-2H-pyrido[3,4-f][1,4]oxazepine-9-carbonitrile.

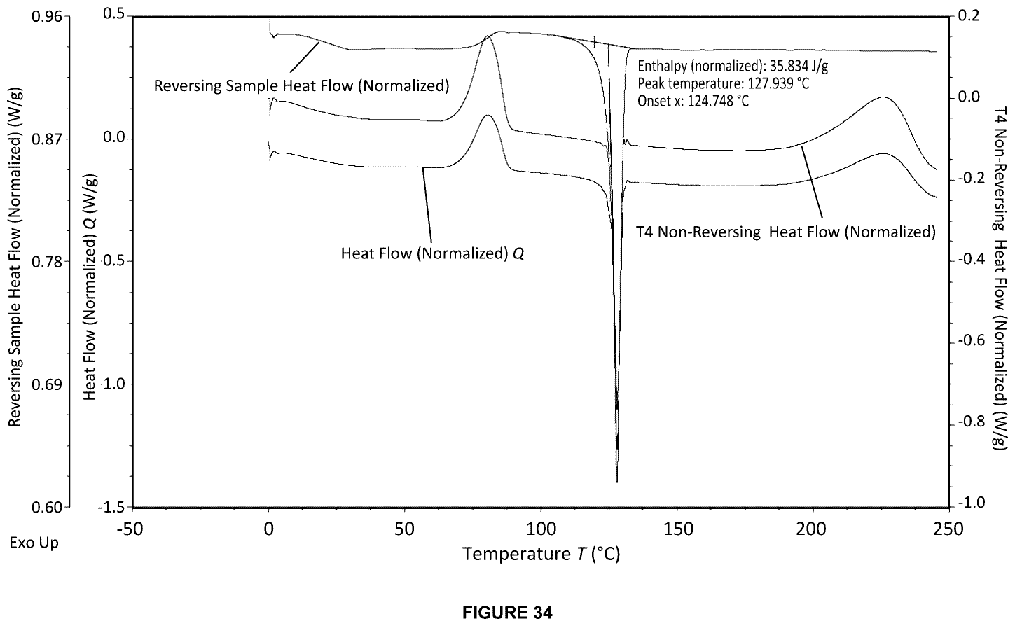

FIG. 35 shows the XRPD spectrum of a substantially amorphous form of 4-(3,3-difluoro-2,2-dimethyl-propanoyl)-3,5-dihydro-2H-pyrido[3,4-f][1,4]oxazepine-9-carbonitrile as described hereinbelow.

FIG. 36 shows the XRPD spectrum of the substantially amorphous form of -(3,3-difluoro-2,2-dimethyl-propanoyl)-3,5-dihydro-2H-pyrido[3,4-f][1,4]oxazepine-9-carbonitrile shown in FIG. 35 after conversion to a crystalline form as described herein.

The details of the disclosure are set forth in the accompanying description below. Although methods and materials similar or equivalent to those described herein can be used in the practice or testing of the present disclosure, illustrative methods and materials are now described. While the disclosure provides illustrated embodiments, it will be understood that they are not intended to limit the invention to those embodiments. On the contrary, the invention is intended to cover all alternatives, modifications, and equivalents, which may be included within the disclosure as defined by the appended claims.

Any section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless otherwise stated, the following terms used in the specification and claims are defined for the purposes of this disclosure and have the following meanings.

### TERMS

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The terms “article of manufacture” and “kit” are used as synonyms.

As used herein, the term “pharmaceutically acceptable carrier” or “pharmaceutically acceptable excipient” or “excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

As used herein, terms “the RIPK1 inhibitor,” “the RIPK1 inhibitor compound,” “the compound of Formula (1),” “Compound (1),” and “the compound,” each refer to 4-(3,3-difluoro-2,2-dimethyl-propanoyl)-3,5-dihydro-2H-pyrido[3,4-f][1,4]oxazepine-9-carbonitrile, having the following structure:

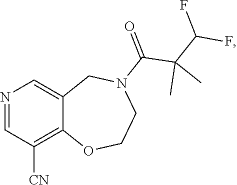 [SMILES: CC(C)(C(=O)N1CCOc2c(C#N)cncc2C1)C(F)F]

or a pharmaceutically acceptable salt thereof.

As used herein, the term “crystalline” or “crystalline solid form,” refers to a solid form which is substantially free of any amorphous solid-state form. In some embodiments, the crystalline solid form is a single solid-state form, e.g. crystalline Form A.

In some embodiments, “substantially free” means less than about 10% w/w, less than about 9% w/w, less than about 8% w/w, less than about 7% w/w, less than about 6% w/w, less than about 5% w/w, less than about 4% w/w, less than about 3% w/w, less than about 2.5% w/w, less than about 2% w/w, less than about 1.5% w/w, less than about 1% w/w, less than about 0.75% w/w, less than about 0.50% w/w, less than about 0.25% w/w, less than about 0.10% w/w, or less than about 0.05% w/w of other crystalline forms of the compound and the amorphous compound. In some embodiments, “substantially free” means an undetectable amount of other crystalline forms of the compound and the amorphous compound.

As used herein, the term “substantially pure” or “substantially crystalline” means that the crystalline form contains at least 90 percent, for example at least 95 percent, such as at least 97 percent, and even at least 99 percent by weight of the indicated crystalline form compared to the total weight of the compound of all forms.

Alternatively, it will be understood that “substantially pure” or “substantially crystalline” means that the crystalline form contains less than 10 percent, for example less than 5 percent, such as less than 3 percent, and even less than 1 percent by weight of impurities, including other polymorphic, solvated or amorphous forms compared to the total weight of the compound of all forms.

As used herein, the term “amorphous” refers to a solid material having no long-range order in the position of its molecules. Amorphous solids are generally supercooled liquids in which the molecules are arranged in a random manner so that there is no well-defined arrangement, e.g., molecular packing, and no long-range order. For example, an amorphous material is a solid material having no sharp characteristic signal(s) in its X-ray power diffractogram (i.e., is not crystalline as determined by XRPD). Instead, one or more broad peaks (e.g., halos) appear in its diffractogram. Broad peaks are characteristic of an amorphous solid.

As used herein, the term “substantially amorphous” refers to a solid material having little or no long-range order in the position of its molecules. For example, substantially amorphous materials have less than 15% crystallinity (e.g., less than 10% crystallinity or less than 5% crystallinity). “Substantially amorphous” includes the descriptor “amorphous,” which refers to materials having no (0%) crystallinity.

The term “modulate” or “modulation” as used herein, means to interact with a target either directly or indirectly so as to alter the activity of the target, including, by way of example only, to enhance the activity of the target, to inhibit the activity of the target, to limit the activity of the target, or to extend the activity of the target.

An “XRPD pattern” or “X-ray powder diffraction pattern” is an x-y graph with diffraction angle (i.e., °2θ) on the x-axis and intensity on the y-axis. The peaks within this pattern may be used to characterize a crystalline solid form. As with any data measurement, there is variability in XRPD data. The data are often represented solely by the diffraction angle of the peaks rather than including the intensity of the peaks because peak intensity can be particularly sensitive to sample preparation (for example, particle size, moisture content, solvent content, and preferred orientation effects influence the sensitivity), so samples of the same material prepared under different conditions may yield slightly different patterns; this variability is usually greater than the variability in diffraction angles. Diffraction angle variability may also be sensitive to sample preparation. Other sources of variability come from instrument parameters and processing of the raw X-ray data: different X-ray instruments operate using different parameters and these may lead to slightly different XRPD patterns from the same solid form, and similarly different software packages process X-ray data differently and this also leads to variability. These and other sources of variability are known to those of ordinary skill in the pharmaceutical arts. Due to such sources of variability, it is usual to assign a variability of about ±0.2°θ to diffraction angles in XRPD patterns.

Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### DETAILED DESCRIPTION OF THE INVENTION

The present disclosure relates to solid forms of 4-(3,3-difluoro-2,2-dimethyl-propanoyl)-3,5-dihydro-2H-pyrido[3,4-f][1,4] oxazepine-9-carbonitrile.

The present disclosure also relates to solid forms of 4-(3,3-difluoro-2,2-dimethyl-propanoyl)-3,5-dihydro-2H-pyrido[3,4-f][1,4] oxazepine-9-carbonitrile that are crystalline. In some embodiments, the crystalline solid form is at least 50% crystalline form, such as at least 60%, at least 70%, at least 80%, at least 90%, at least 95%, at least 96%, at least 97%, at least 98%, at least 99%, or 100% crystalline.

The present disclosure still further relates to a solid form of 4-(3,3-difluoro-2,2-dimethyl-propanoyl)-3,5-dihydro-2H-pyrido[3,4-f][1,4] oxazepine-9-carbonitrile that is characterized as crystalline Form A. In some embodiments, the crystalline solid form characterized as crystalline Form A is at least 50% crystalline form, such as at least 60%, at least 70%, at least 80%, at least 90%, at least 95%, at least 96%, at least 97%, at least 98%, at least 99%, or 100% crystalline.

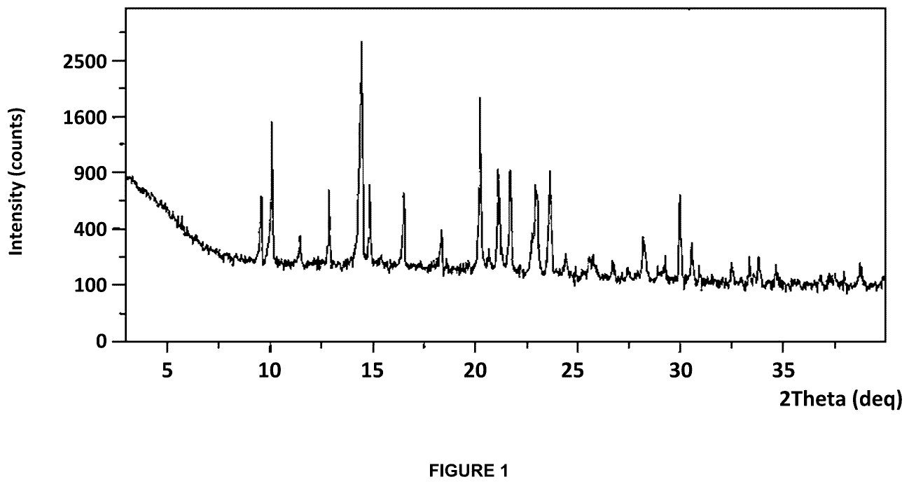

In some embodiments, crystalline Form A has an X-ray powder diffraction (XRPD) pattern derived using Cu (Kα) radiation comprising three, four, five, six, seven or more peaks, in term of 2-theta degrees, chosen from peaks at about 10.1±0.2, 14.3±0.2, 14.8±0.2, 16.4±0.2, 18.2±0.2, 20.1±0.2, 21.0±0.2,21.6±0.2, 22.8±0.2, 23.5±0.2, 28.1±0.2, 29.8±0.2. In some embodiments, the solid form of crystalline Form A has an XRPD pattern derived using Cu (Kα) radiation, in term of 2-theta degrees, having peaks at about 14.3±0.2, 20.1±0.2, 21.6±0.2, 22.8±0.2, and 23.5±0.2. In some embodiments, the solid form of crystalline Form A has an X-ray powder diffraction pattern that is substantially in accordance with that shown in FIG. 1.

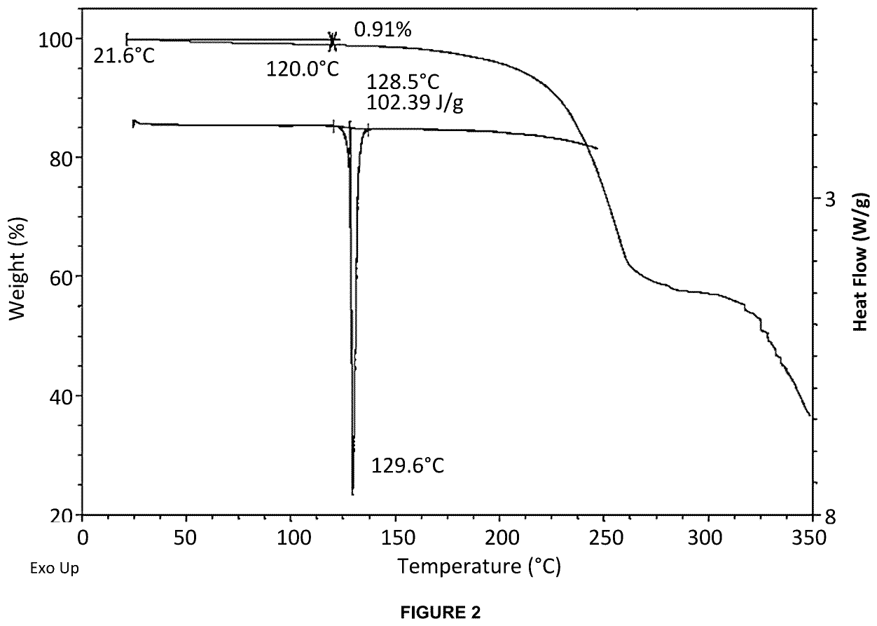

In some embodiments, the solid form of crystalline Form A is characterized by a differential scanning calorimetry (DSC) curve with an onset at about 128.5° C. and an endothermic peak at 129.6° C. In some embodiments, the solid form of crystalline Form A is characterized by a Thermogravimetric Analysis (TGA) profile with an about 0.91% w/w loss from about 21.6° C. to about 120° C. In some embodiments, the solid form of crystalline Form A is characterized by a DCS/TGA profile substantially in accordance with that shown in FIG. 2.

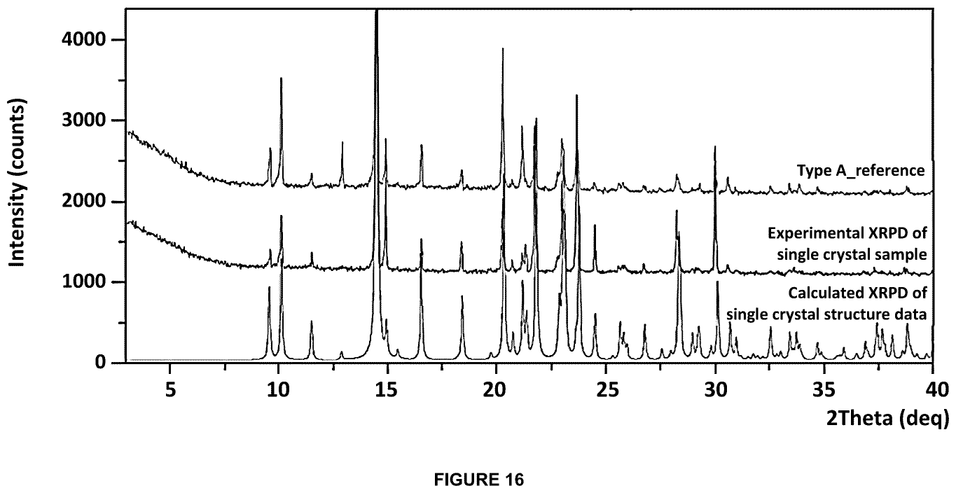

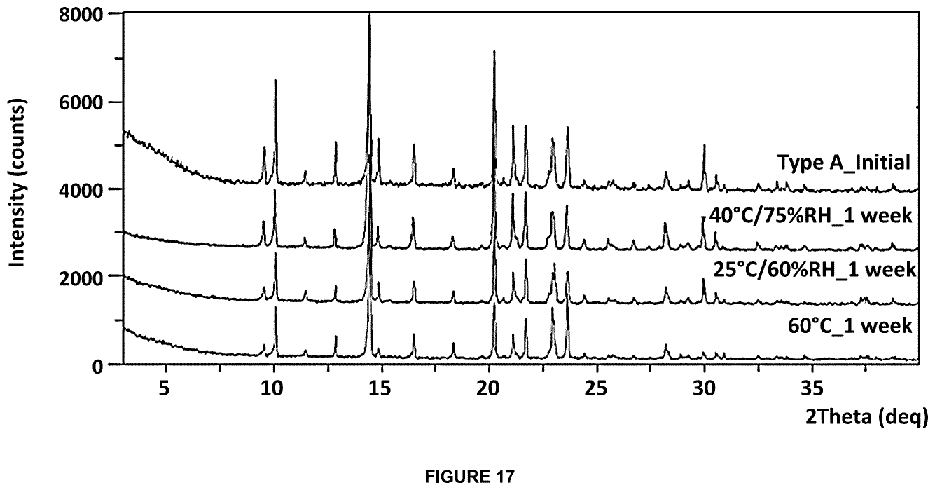

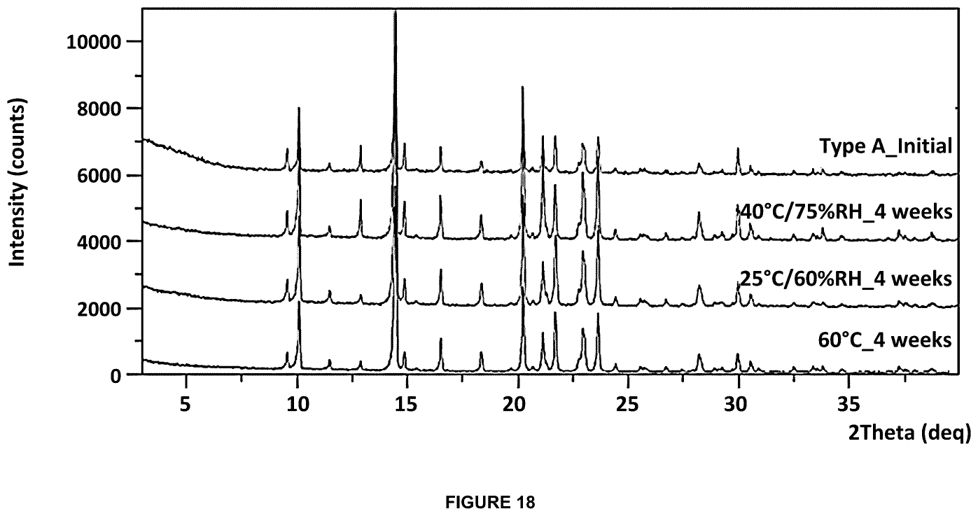

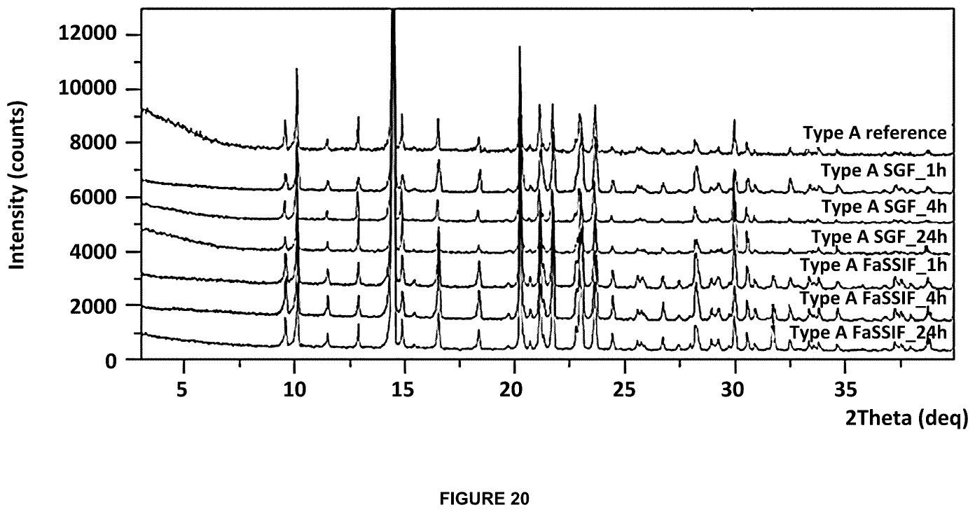

In some embodiments, the solid form of crystalline Form A is characterized by an X-ray powder diffraction pattern that is substantially in accordance with any of those shown in FIG. 16, 17, 18, or 20. In some embodiments, the solid form of crystalline Form A is characterized by an X-ray powder diffraction pattern that is substantially in accordance with FIG. 16. In some embodiments, the solid form of crystalline Form A is characterized by an X-ray powder diffraction pattern that is substantially in accordance with FIG. 17. In some embodiments, the solid form of crystalline Form A is characterized by an X-ray powder diffraction pattern that is substantially in accordance with FIG. 18. In some embodiments, the solid form of crystalline Form A is characterized by an X-ray powder diffraction pattern that is substantially in accordance with FIG. 20.

In some embodiments, crystalline Form A is characterized by at least two of:
a) an X-ray powder diffraction (XRPD) pattern substantially in accordance with that shown in FIG. 1;
b) an X-ray powder diffraction (XRPD) pattern derived using Cu (Kα) radiation comprising three, four, five, six, seven or more peaks, in term of 2-theta degrees, at about 10.1±0.2, 14.3±0.2, 14.8±0.2, 16.4±0.2, 18.2±0.2, 20.1±0.2, 21.0±0.2, 21.6±0.2, 22.8±0.2, 23.5±0.2, 28.1±0.2, 29.8±0.2;
c) a DSC/TGA profile substantially the same as shown in FIG. 2;
d) a Differential Scanning calorimetry (DSC) thermogram having an onset at about 128.5° C. and a peak at about 129.6° C.;
e) a TGA profile with an about 0.91% w/w loss from about 21.6° C. to about 120° C.;
f) an X-ray powder diffraction pattern that is substantially in accordance with any of those shown in FIG. 16;
g) an X-ray powder diffraction pattern that is substantially in accordance with any of those shown in FIG. 17;
h) an X-ray powder diffraction pattern that is substantially in accordance with any of those shown in FIG. 18; or
i) an X-ray powder diffraction pattern that is substantially in accordance with any of those shown in FIG. 20.

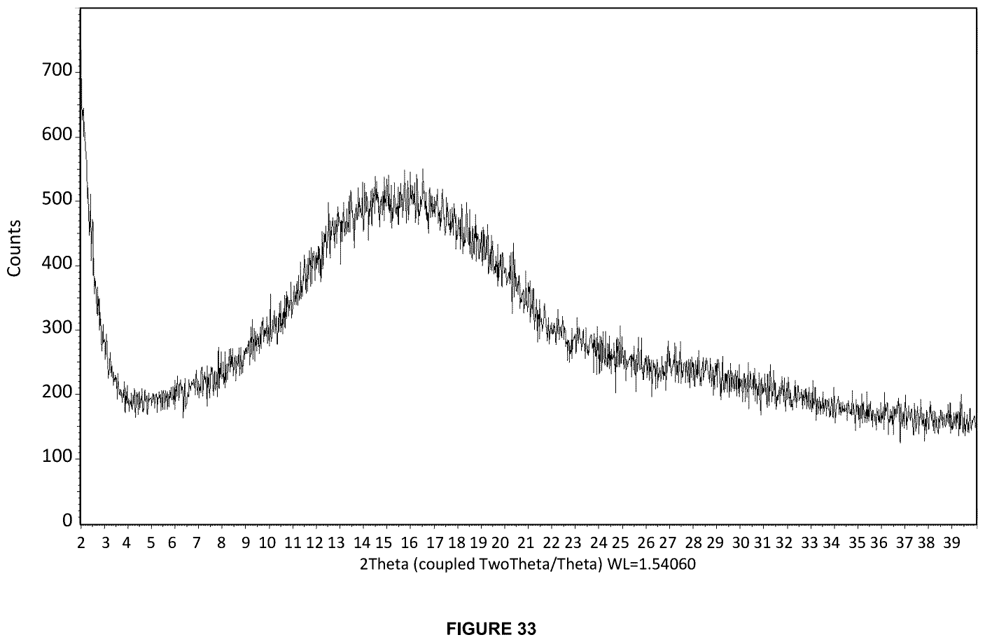

The present disclosure further relates to solid forms of 4-(3,3-difluoro-2,2-dimethyl-propanoyl)-3,5-dihydro-2H-pyrido[3,4-f][1,4]oxazepine-9-carbonitrile, characterized as amorphous. In some embodiments, the solid amorphous form of 4-(3,3-difluoro-2,2-dimethyl-propanoyl)-3,5-dihydro-2H-pyrido[3,4-f][1,4] oxazepine-9-carbonitrile is characterized by at least one of:
a) an X-ray powder diffraction (XRPD) pattern substantially in accordance with that shown in FIG. 33; or
b) a Differential Scanning Calorimetry (DSC) thermogram having an onset at about 124.7° C. and a peak at about 127.9° C.

The present disclosure also relates to pharmaceutical compositions comprising any of the solid forms disclosed herein and a pharmaceutically acceptable carrier.

The present disclosure still further relates to a method of treating a disease and/or condition mediated by RIPK1 in a patient in need thereof, comprising administering to the patient an effective amount of any of the solid forms disclosed herein.

The present disclosure relates to a solid form as disclosed herein for use in treating a disease and/or condition mediated by RIPK1 in a patient in need thereof.

The present disclosure also relates to use of any solid form disclosed herein in the manufacture of a medicament for treating a disease involving mediation of the RIPK1 receptor.

In certain embodiments, the disease or disorder is inflammatory bowel disease, Crohn's disease, ulcerative colitis, psoriasis, retinal detachment, retinitis pigmentosa, macular degeneration, pancreatitis, atopic dermatitis, rheumatoid arthritis, spondyloarthritis, gout, SoJIA, systemic lupus erythematosus, Sjogren's syndrome, systemic scleroderma, anti-phospholipid syndrome, vasculitis, osteoarthritis, non-alcohol steatohepatitis, alcohol steatohepatitis, autoimmune hepatitis, autoimmune hepatobiliary diseases, primary sclerosing cholangitis, nephritis, Celiac disease, autoimmune ITP, transplant rejection, ischemia reperfusion injury of solid organs, sepsis, systemic inflammatory response syndrome, cerebrovascular accident, myocardial infarction, Huntington's disease, Alzheimer's disease, Parkinson's disease, allergic diseases, asthma, atopic dermatitis, multiple sclerosis, type I diabetes, Wegener's granulomatosis, pulmonary sarcoidosis, Behcet's disease, interleukin-1 converting enzyme associated fever syndrome, chronic obstructive pulmonary disease, tumor necrosis factor receptor-associated periodic syndrome, or peridontitis.

In certain embodiments, the disease or disorder is trauma, ischemia, stroke, cardiac infarction, infection, lysosomal storage disease, Gaucher's disease, Krabbe disease, Niemann-Pick disease, sepsis, Parkinson's disease, Alzheimer's disease, amyotrophic lateral sclerosis (ALS/Lou Gehrig's Disease), Huntington's disease, HIV-associated dementia, retinal degenerative disease, glaucoma, age-related macular degeneration, rheumatoid arthritis, psoriasis, psoriatic arthritis or inflammatory bowel disease.

In certain embodiments, the disease or disorder is Alzheimer's disease, ALS, Friedreich's ataxia, Huntington's disease, Lewy body disease, Parkinson's disease, or spinal muscular atrophy. In certain embodiments, the disease or disorder is brain injury, spinal cord injury, dementia, stroke, Alzheimer's disease, ALS, Parkinson's disease, Huntington's disease, multiple sclerosis, diabetic neuropathy, poly glutamine (polyQ) diseases, stroke, Fahr disease, Menke's disease, Wilson's disease, cerebral ischemia, or a prion disorder.

The present disclosure also provides compounds and pharmaceutical compositions that are useful in inhibiting RIPK1.

Each embodiment described herein may be taken alone or in combination with any one or more other embodiments.

### SOLID FORMS

Compound of Formula (1) refers to 4-(3,3-difluoro-2,2-dimethyl-propanoyl)-3,5-dihydro-2H-pyrido[3,4-f][1,4]oxazepine-9-carbonitrile, which has the chemical structure shown below:

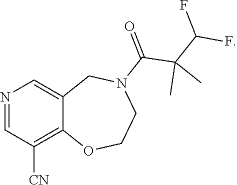 [SMILES: CC(C)(C(=O)N1CCOc2c(C#N)cncc2C1)C(F)F]

In some embodiments provided herein, Compound of Formula (1) is crystalline.

In some embodiments, the crystallinity of a solid form is characterized by X-Ray Powder Diffraction (XRPD).

In some embodiments, the crystallinity of a solid form is determined by differential scanning calorimeter (DSC).

In some embodiments, the crystallinity of a solid form is determined by thermogravimetric analysis (TGA) in combination with XRPD and/or DSC.

### PREPARATION OF COMPOUND OF FORMULA (1)

Compound (1) described herein may be made as described below:

The synthetic route is set forth below:

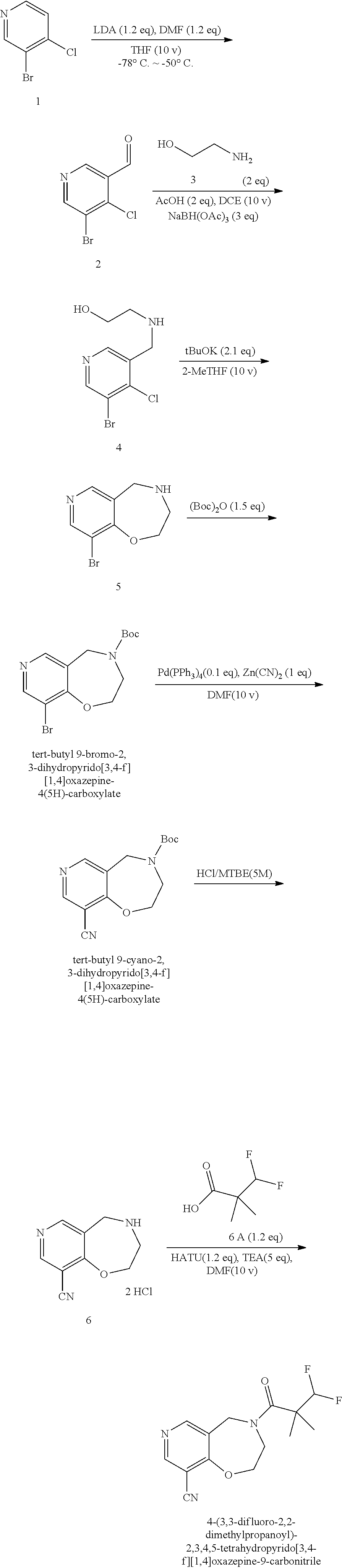

Compound 2 was made as follows:

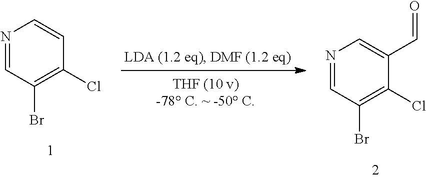 [SMILES: Clc1ccncc1Br ; O=Cc1cncc(Br)c1Cl]

Two reactions were carried out in parallel. To a solution of diisopropylamine (1.23 kg, 12.2 mol, 1.72 L, 1.2 eq) in THF (10 L) was added n-BuLi (2.5 M, 4.86 L, 1.2 eq) at-30° C. under N2, and the mixture was stirred at −30° C. for 30 min. Then the mixture was added to a solution of compound 1 (1950 g, 10.13 mol, 1 eq) in THF (16 L) at −78° C. under N2, and the reaction was stirred at −78° C. for 2.5 h. DMF (889 g, 12.2 mol, 936 mL, 1.2 eq) was added to the reaction mixture at −78° C., and the resulting mixture was stirred at −50° C. for 1 h. TLC (PE: EtOAc=5:1) indicated compound 1 was consumed completely and one new spot (Rf_{R1}=0.55, Rf_{P1}=0.50) formed. The reaction was clean according to TLC. The reaction mixture was quenched by addition of sat. aq. NH₄Cl (10 L), and the aqueous was extracted with EtOAc (5 L). The combined organic layers were washed with brine (10 L×1), dried over Na₂SO₄, filtered and concentrated under reduced pressure to give a residue. The crude product was dissolved with EtOAc (16 L), and filtered. The organic layers were washed with 1M HCl solution (2 L), and brine (2 L). The two batches were combined, dried over anhydrous Na₂SO₄, filtered and concentrated in vacuo to give compound 2 (2800 g, 12.7 mol, 62.7% yield) as a yellow solid without further purification.

¹H NMR: 400 MHZ CDClδ 10.46 (s, 1H), 8.92 (d, J=1.6 Hz, 2H).

Compound 4 was made as follows:

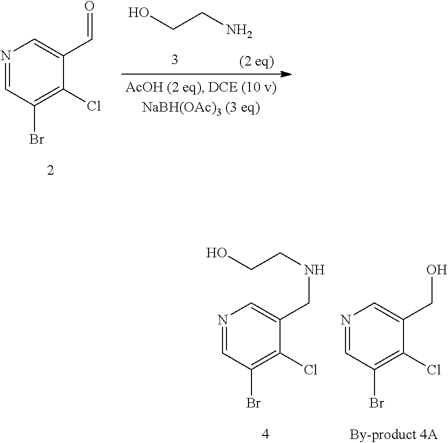

Two reactions were carried out in parallel. To a solution of compound 2 (1400 g, 6.35 mol, 1 eq) in DCE (14 L) was added compound 3 (776 g, 12.7 mol, 768 mL, 2 eq), followed by AcOH (1.14 kg, 19.1 mol, 1.09 L, 3 eq) at 0˜15° C. The mixture was stirred at 25° C. for 1 hr under N₂ atmosphere. NaBH(OAc)₃ (2.69 kg, 12.7 mol, 2 eq) was then added at 0˜15° C. and the reaction mixture was stirred at 25° C. for 12 h. TLC (DCM: MeOH=20:1) indicated compound 2 was consumed completely and one new spot (Rf_{P1}=0.33) formed. LC-MS showed no compound 2 remained. Several new peaks were shown on LC-MS (Retention time=1.2 min) and one main peak with desired mass was detected. The two batches were combined together for workup. The reaction mixture was diluted with water (10 L) and stirred 30 min. The layers were separated and the aqueous layer was extracted with DCM (2 L). The aqueous was added aqueous NaOH (5M) till pH to 9˜10. The aqueous was extracted with DCM (3×8 L). The combined organic layers were washed with brine (1×2 L), dried over Na₂SO₄, filtered and concentrated under reduced pressure to give compound 4 (2000 g, 7.28 mol, 57.34% yield, 96.7% purity) as a yellow solid, which was used in the next step without further purification.

¹H NMR: 400 MHZ CDCl₃ δ 8.66 (s, 1H), 8.49 (s, 1H), 3.97 (s, 2H), 3.61-3.78 (m, 3H), 2.72-2.86 (m, 2H).

Compound 5 was made as follows:

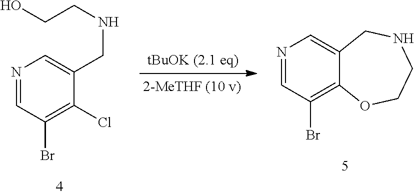 [SMILES: OCCNCc1cncc(Br)c1Cl ; Brc1cncc2c1OCCNC2]

To a solution of compound 4 (1050 g, 3.95 mol, 1 eq) in 2-methyltetrahydrofuran (10 L) was added t-BuOK (932 g, 8.30 mol, 2.1 eq) at 0˜20° C. The reaction mixture was stirred at 25° C. for 2 hr. LC-MS and HPLC showed that no compound 4 remained. Several new peaks were shown on LC-MS (Retention time=1.36 min) and one main peak with desired mass was detected. The reaction mixture was used in the next step directly.

Preparation of tert-butyl 9-bromo-2,3-dihydropyrido[3,4-f][1,4]oxazepine-4(5H)-carboxylate:

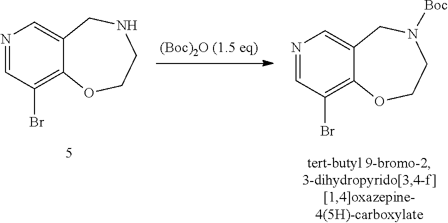 [SMILES: Brc1cncc2c1OCCNC2 ; CC(C)(C)OC(=O)N1CCOc2c(Br)cncc2C1]

Two reactions were carried out in parallel. Boc₂O (1.17 kg, 5.37 mol, 1.23 L, 1.5 eq) was added to the reaction mixture of compound 5 (819.55 g, 3.58 mol, 1 eq) at 25° C., and the mixture was stirred at 25° C. for 16 h under N₂ atmosphere. LC-MS showed no compound 5 remained. Several new peaks were shown on LC-MS (Retention time=1.31 min) and one main peak with desired mass was detected. The two batches were combined together. The reaction mixture was added water (15 L) at 15° C., and the aqueous was extracted with EtOAc (3 L×2). The combined organic layers were washed with brine (5 L), dried over Na₂SO₄, filtered and concentrated. The residue was purified by column chromatography (SiO₂, Petroleum ether/Ethyl acetate=15:1 to 1:1, PE:EtOAc=3:1, Rf_{P1}=0.43) to give tert-butyl 9-bromo-2,3-dihydropyrido[3,4-f][1,4] oxazepine-4(5H)-carboxylate (1520 g, 100% purity) as an off-white solid.

¹H NMR: 400 MHz CDCl₃ δ 8.59 (br s, 1H), 8.24-8.38 (m, 1H), 4.45-4.64 (m, 2H), 4.26 (br s, 2H), 3.84-3.91 (m, 2H), 1.43 (s, 9H).

Procedure for preparation of tert-butyl 9-cyano-2,3-dihydropyrido[3,4-f][1,4]oxazepine-4(5H)-carboxylate:

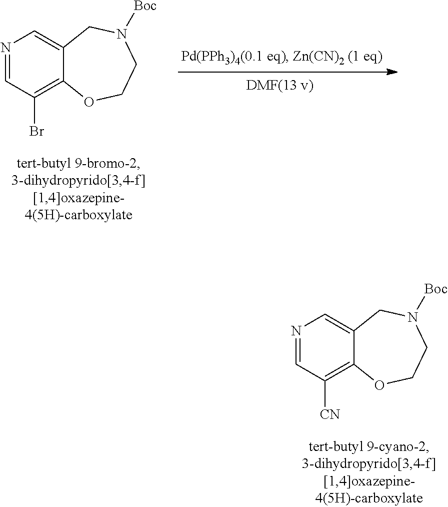 [SMILES: CC(C)(C)OC(=O)N1CCOc2c(Br)cncc2C1 ; CC(C)(C)OC(=O)N1CCOc2c(C#N)cncc2C1]

Five reactions were carried out in parallel. A mixture of tert-butyl 9-bromo-2,3-dihydropyrido[3,4-f][1,4]oxazepine-4(5H)-carboxylate (200 g, 608 mmol, 1 eq), Pd(PPh₃)₄ (70.2 g, 60.8 mmol, 0.1 eq), Zn(CN)₂(74.9 g, 638 mmol, 40.5 mL, 1.05 eq) in DMF (2 L) was degassed and purged with N₂ for 3 times, and the mixture was stirred at 110° C. for 16 hr under N₂ atmosphere. LC-MS showed no tert-butyl 9-bromo-2,3-dihydropyrido[3,4-f][1,4]oxazepine-4(5H)-carboxylate remained. Several new peaks were shown on LC-MS (Retention time=1.36 min) and one main peak with desired mass was detected. The five batches were combined together for workup. The reaction mixture was poured into H₂O (20 L) slowly and then the mixture was filtered. The filtrate was extracted with MTBE (10 L×5). The organic phase was washed with brine (500 mL), dried over anhydrous Na₂SO₄, concentrated in vacuum to give a residue. The residue was purified by column chromatography (SiO₂, Petroleum ether:Ethyl acetate=3:1 to 1:1, PE:EtOAc=1:1, Rf_{P1}=0.23) to give tert-butyl 9-cyano-2,3-dihydropyrido[3,4-f][1,4]oxazepine-4(5H)-carboxylate (720 g, 2.45 mol, 80.7% yield, 93.7% purity) was obtained as an off-white solid.

¹H NMR: 400 MHz CDCl₃ δ 8.62 (br s, 1H), 8.45 (br s, 1H), 4.40-4.70 (m, 3H), 3.84-3.91 (m, 2H), 1.34-1.46 (m, 9H).

Procedure for preparation of compound 6:

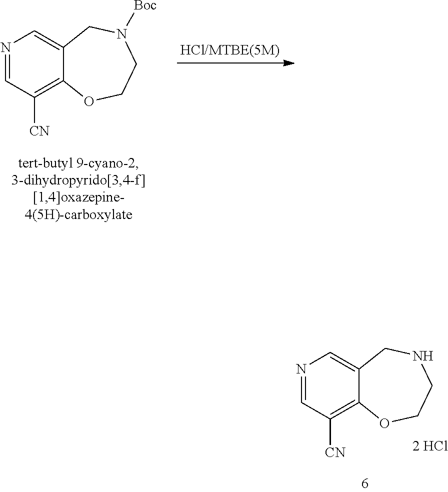 [SMILES: CC(C)(C)OC(=O)N1CCOc2c(C#N)cncc2C1 ; N#Cc1cncc2c1OCCNC2]

Four reactions were carried out in parallel. To a mixture of tert-butyl 9-cyano-2,3-dihydropyrido[3,4-f][1,4]oxazepine-4(5H)-carboxylate (180 g, 654 mmol, 1 eq) in MTBE (1500 mL) was added HCl/MTBE (5 M, 700 mL) drop-wise at 25° C. under N₂. The mixture was stirred at 25° C. for 2 hr. LC-MS showed no tert-butyl 9-cyano-2,3-dihydropyrido [3,4-f][1,4]oxazepine-4(5H)-carboxylate remained. Several new peaks were shown on LC-MS (Retention time=0.46 min) and one main peak with desired mass was detected. The four batches were combined together for workup. The solid was collected by filtration to give compound 6 (620 g, 2.48 mol, 95% yield, 99.4% purity, 2HCl) as an off-white solid.

¹H NMR: 400 MHz DMSO-d₆ δ 10.17 (br s, 2H), 8.89 (s, 1H), 8.74 (s, 1H), 4.54-4.74 (m, 2H), 4.54 (s, 2H), 3.60 (s, 2H).

Procedure for preparation of 4-(3,3-difluoro-2,2-dimethylpropanoyl)-2,3,4,5-tetrahydropyrido[3,4-f][1,4]oxazepine-9-carbonitrile:

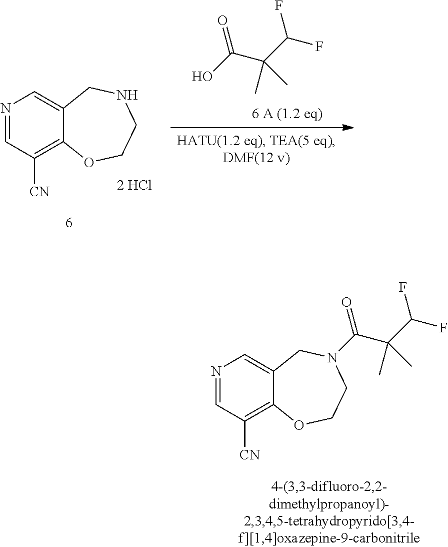

Two reactions were carried out in parallel. To a solution of compound 6A (50.1 g, 363 mmol, 1.2 eq), Et₃N (153 g, 1.51 mol, 210 mL, 5 eq) and compound 6 (75 g, 302 mmol, 1 eq, 2HCl) in DMF (750 mL) was added HATU (138 g, 363 mmol, 1.2 eq) in portions at 0° C. under N₂ atmosphere. The mixture was stirred at 25° C. for 2 hr under N₂ atmosphere. LC-MS showed that no compound 6 remained. Several new peaks were shown on LC-MS (Retention time=1.18 min) and one main peak with desired mass was detected. The two batches were combined together for workup. The reaction mixture was added water (2 L) at 0° C., and the aqueous was extracted with EtOAc 3 L (1 L×3). The combined organic layers were washed with brine (500 mL×3), dried over Na₂SO₄, filtered and concentrated under reduced pressure to give a residue. The crude mixture was dissolved in EtOAc (2 L), and added the Pd-removal silica gel (10 g). The mixture was stirred at 25° C. for 2 hr, then filtered and concentrated under reduced pressure to give a residue. The residue was purified by column chromatography (SiO₂, Heptane:Ethyl acetate=5:1 to 1:1). Then the crude product (180 g) was added EtOAc (200 mL) and the mixture was heated at reflux to provide a clear solution. The solution was filtered under vacuum. The resulting mixture was added n-heptane (100 mL) drop-wise and stirred at 25° C. for 2 hr. Then white solid had crystallized. The white solid was collected by filtration to give 4-(3,3-difluoro-2,2-dimethylpropanoyl)-2,3,4,5-tetrahydropyrido[3,4-f][1,4]oxazepine-9-carbonitrile (85 g, 287.28 mmol, 47.52% yield, 99.8% purity) as a white solid.

¹H NMR: 400 MHz DMSO-d₆ δ 8.72 (s, 1H), 8.67 (s, 1H), 6.22 (t, J=56.4 Hz, 1H), 4.84 (br s, 2H), 4.73 (t, J=5.2 Hz, 2H), 4.02 (br s, 2H), 1.26 (s, 6H).

### PHARMACEUTICAL COMPOSITIONS

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or incombination with pharmaceutically acceptable carriers, excipients or diluents, in a pharmaceutical composition. Administration of the compounds and compositions described herein can be affected by any method that enables delivery of the compounds to the site of action.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent.

In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally containgum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

It should be understood that in addition to the ingredients particularly mentioned above, the compounds and compositions described herein may include other agents conventional in the art having regard to the type of formulation in question, for example those suitable for oral administration may include flavoring agents.

Herein is also provided a pharmaceutical composition comprising a crystalline Form A of the Compound of Formula (1) and a pharmaceutically acceptable carrier. In one aspect, in said pharmaceutical composition, said crystalline Form A is substantially pure and substantially free of other crystalline forms of the Compound of Formula (1). In another aspect, in said pharmaceutical composition, said crystalline Form A is at least 90 percent by weight of all forms.

### METHODS OF DOSING AND TREATMENT REGIMENS

The specific dose level of a compound of the present application for any particular patient will depend upon a variety of factors including the activity of the specific compound employed, the age, body weight, general health, sex, diet, time of administration, route of administration, and rate of excretion, drug combination and the severity of the particular disease in the patient undergoing therapy. For example, a dosage may be expressed as a number of milligrams of a compound described herein per kilogram of the patient's body weight (mg/kg). Dosages of between about 0.1 and 150 mg/kg may be appropriate. In certain embodiments, about 0.1 and 100 mg/kg may be appropriate. In other embodiments a dosage of between 0.5 and 60 mg/kg may be appropriate. Normalizing according to the patient's body weight is particularly useful when adjusting dosages between patients of widely disparate size, such as occurs when using the drug in both children and adult humans or when converting an effective dosage in a non-human patient such as dog to a dosage suitable for a human patient.

The daily dosage may also be described as a total amount of a compound disclosed herein administered per dose or per day. Daily dosage of a compound disclosed herein may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

When administered orally, the total daily dosage for a human patient may be between 1 mg and 1,000 mg, between about 1,000-2,000 mg/day, between about 10-500 mg/day, between about 50-300 mg/day, between about 75-200 mg/day, or between about 100-150 mg/day.

In certain embodiments, the method comprises administering to the patient an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

Herein is also provided a method of treating a disease and condition mediated by RIPK1 in a patient in need thereof, comprising administering to the patient an effective amount of the crystalline Form A of the Compound of Formula (1).

Herein is also provided the crystalline Form A of the Compound of Formula (1) for use as a medicine, for use as an inhibitor RIPK1 receptor, and for use in the treatment of various diseases wherein RIPK1 receptor is involved.

Herein is also provided use of the crystalline Form A of the Compound of Formula (1) for the manufacture of a medicament for treating a disease involving inhibition of RIPK1 receptor.

### ARTICLES OF MANUFACTURE AND KITS

Disclosed herein, in certain embodiments, are kits and articles of manufacture for use with one or more methods described herein. In some embodiments, additional component of the kit comprises a package or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastic.

The articles of manufacture provided herein contain packaging materials. Examples of pharmaceutical packaging materials include, but are not limited to, bottles, tubes, bags, containers, and any packaging material suitable for a selected formulation and intended mode of use.

For example, the container(s) include one or more of the compounds described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### ABBREVIATIONS

The following abbreviations may be relevant for this application.

| | |
|---|---|
| ACN or MeCN: | acetonitrile; |
| CAN: | ceric ammonium nitrate; |
| CPME: | cyclopentyl methyl ether; |
| DCM: | dichloromethane; |
| DMSO: | dimethylsulfoxide; |
| DMAc: | N,N-Dimethylacetamide; |
| DSC: | differential scanning calorimetry; |
| DVS: | dynamic vapor sorption; |
| Et: | ethyl; |
| EtOAc: | ethyl acetate; |
| EtOH: | ethanol; |
| equiv or eq.: | equivalents; |
| FaSSIF: | fasted state simulated intestinal fluid; |
| FeSSIF: | fed state simulated intestinal fluid; |
| FTIR: | Fourier transform infrared; |
| h or hr: | hour; |
| hrs: | hours; |
| HPLC: | high-performance liquid chromatography; |
| IPA: | isopropyl alcohol; |
| IPAc: | isopropyl acetate; |
| KCl: | potassium chloride; |
| LC-MS or LCMS or LC/MS: | liquid chromatography-mass spectrometry; |
| LiCl: | lithium chloride; |
| M: | molar; |
| Me: | methyl; |
| MeOH: | methanol; |
| MeOAc: | methyl acetate; |
| Mg(NO₃)₂: | magnesium nitrate; |
| MIBK: | methyl isobutyl ketone; |
| MTBE: | methyl tert-butyl ether; |
| mins or min: | minutes; |
| N₂: | nitrogen; |
| n-PrOAc: | n-propyl acetate; |
| NMR: | nuclear magnetic resonance; |
| RH: | relative humidity; |
| rt or RT: | room temperature; |
| SCXRD: | single crystal x-ray diffraction; |
| SGF: | simulated gastric fluid; |
| TFA: | trifluoroacetic acid; |
| TGA: | thermogravimetric analysis; |
| THF: | tetrahydrofuran; |
| 2-MeTHF: | 2-methyltetrahydroguran; |
| vol: | volume; |
| w/w: | weight ratio; and |
| XRPD: | X-ray powder diffraction. |

The following examples are provided for illustrative purposes only and not to limit the scope of the claims provided herein.

### INSTRUMENTS AND METHODS

### 1-XRPD

For XRPD analysis, PANalytical Empyrean and X′ Pert3 X-ray powder diffractometer were used. The XRPD parameters used are listed in Table A.

TABLE A
Parameters for XRPD test

| Parameters | Empyrean | X′ Pert3 | X′ Pert3 |
|---|---|---|---|
| X-Ray | Cu, Kα; | Cu, Kα; | Cu, Kα; |
| wavelength | Kα1 (Å): 1.540598 | Kα1 (Å): 1.540598 | Kα1 (Å): 1.540598 |
| | Kα2 (Å): 1.544426 | Kα2 (Å): 1.544426 | Kα2 (Å): 1.544426 |
| | intensity ratio | intensity ratio | intensity ratio |
| | Kα2/Kα1: 0.50 | Kα2/Kα1: 0.50 | Kα2/Kα1: 0.50 |
| X-Ray tube setting | 45 kV, 40 mA | 45 kV, 40 mA | 45 kV, 40 mA |
| Divergence slit | Automatic | ⅛° | ⅛° |
| Scan mode | Continuous | Continuous | Continuous |
| Scan range (2θ/°) | 3°~40° | 3°~40° | 3°~40° |
| Step size (2θ/°) | 0.0167° | 0.0263° | 0.0263° |
| Scan step time (s) | 17.780 | 46.665 | 39.525 |
| Test time (s) | About 5 mins 30 s | About 5 mins | 4 mins 27 s |

### 2-TGA and DSC

TGA data were collected using a TA Q500/Q5000 TGA from TA Instruments. DSC was performed using a TA Q200/Q2000 DSC from TA Instruments. Detailed parameters used are listed in Table B.

TABLE B
Parameters for TGA and DSC test

| Parameters | TGA | DSC |
|---|---|---|
| Method | Ramp | Ramp |
| Sample pan | Aluminum, open | Aluminum, crimped/open |
| Temperature | RT-desired | 25° C.-desired |
| | temperature | temperature |
| Heating rate | 10° C./min | 10° C./min |
| Purge gas | N₂ | N₂ |

### 3-DVS

DVS was measured via a SMS (Surface Measurement Systems) DVS Intrinsic. The relative humidity at 25° C. were calibrated against deliquescence point of LiCl, Mg(NO₃)₂ and KCl. Parameters for DVS test were listed in Table C.

TABLE C
Parameters for DVS test

| Parameters | DVS | |
|---|---|---|
| Temperature | 25° | C. |
| Sample size | 10~20 | mg |
| Gas and flow rate | N₂, 200 | mL/min |
| dm/dt | 0.002%/min | |
| Min. dm/dtstabilityduration | 10 | min |
| Max. equilibrium time | 180 | min |

| | |
|---|---|
| RH range | 95% RH-0% RH-95% RH |
| RH step size | 10% (90% RH-0% RH-90% RH) |
| | 5% (95% RH-90% RH and 90% |
| | RH-95% RH) |

### 5-HPLC

Agilent HPLC was utilized and detailed chromatographic conditions for purity and solubility measurement are listed in Table D.

TABLE D
Chromatographic conditions and parameters
for purity/solubility test

| Parameters | Agilent 1260 DAD Detector | |
|---|---|---|
| Column | Phenomenex Gemini C18, 150 × 4.6 mm, 3 μm | |
| Mobile phase | A: 0.037% TFA in Water | |
| | B: 0.018% TFA in Acetonitrile | |
| Gradient table | Time (min) | % B |
| | 0.00 | 10 |
| | 0.10 | 10 |
| | 7.00 | 80 |
| | 10.00 | 100 |
| | 10.01 | 10 |
| | 15.00 | 10 |

| | |
|---|---|
| Run time | 15.0 min |
| Post time | 0.0 min |
| Flow rate | 0.8 mL/min |
| Injection volume | 5 μL |
| Detector wavelength | UV at 220 nm |
| Column temperature | 40° C. |
| Sampler temperature | RT |
| Diluent | Acetonitrile/Water (1:1) |

### 6-LC-MS

Shimadzu LC-MS was utilized and detailed conditions for measurement are listed in Table E.

TABLE E
Conditions and parameters for LC-MS test

| Parameters | Shimadzu-LC-MS 2020 | |
|---|---|---|
| Column | Sepax BR-C18 4.6 * 50 mm, 3 um | |
| Mobile Phrase | A: 0.1% FA in Water | |
| | B: Acetonitrile | |
| Gradient table | Time (min) | % B |
| | 0.00 | 20 |
| | 0.20 | 20 |
| | 2.00 | 80 |
| | 4.80 | 80 |
| | 5.00 | 20 |
| | 5.50 | 20 |

| | |
|---|---|
| Run time | 5.50 min |
| Flow rate | 1.0 mL/min |
| Injection volume | 0.4 μL |
| Detector wavelength | UV at 220/254 nm |
| Column temperature | 40° C. |
| Sampler temperature | RT |
| Ion source for MS | ESI |

### 7-PLM

PLM images were captured using Axio Lab A1 upright microscope with ProgRes® CT3 camera at RT.

### 8-pKa

The pKa was measured by a Sirius pKa log P/D tester (model: T3) with a UV detector (UV metric method) using MeOH as solvent.

### EXAMPLE 1—CHARACTERIZATION OF COMPOUND (1) STARTING MATERIAL

Compound (1), made as described herein, was characterized by XRPD, TGA, DSC, PLM, DVS and HPLC purity prior to undergoing polymorph screening.

As displayed in FIG. 1, XRPD revealed that the sample was crystalline and thus named as Form A. Peaks identified in FIG. 1 include those listed in Table 1.

TABLE 1
XRPD Peak list of Form A

| Pos. [°2Th.] (±0.2) | d-spacing [Å] | Rel. Int. [%] |
|---|---|---|
| 10.0 | 8.84 | 10.1 |
| 14.3 | 6.17 | 100.0 |
| 14.8 | 5.99 | 9.5 |
| 16.4 | 5.40 | 13.4 |
| 18.2 | 4.9 | 9.7 |
| 20.1 | 4.4 | 40.5 |
| 21.0 | 4.2 | 25.2 |
| 21.6 | 4.1 | 44.9 |
| 22.8 | 3.9 | 55.8 |
| 23.5 | 3.8 | 33.0 |
| 28.1 | 3.2 | 27.0 |
| 29.8 | 3.0 | 11.9 |

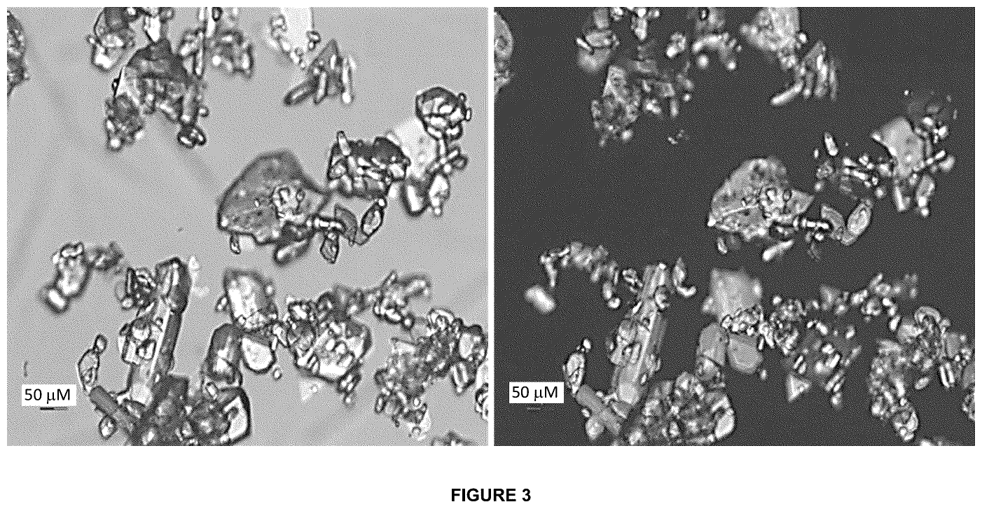

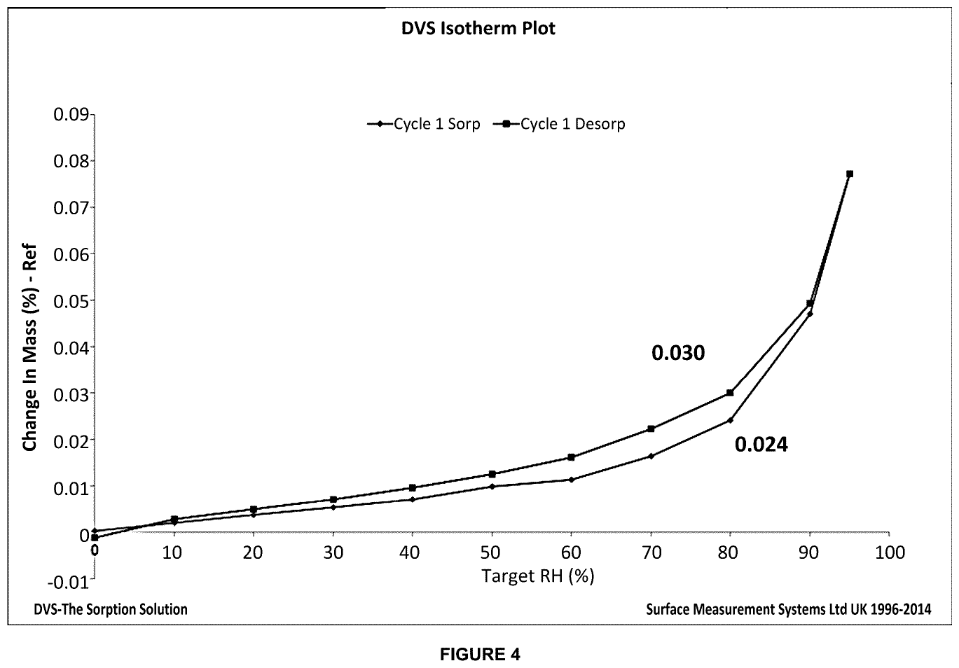

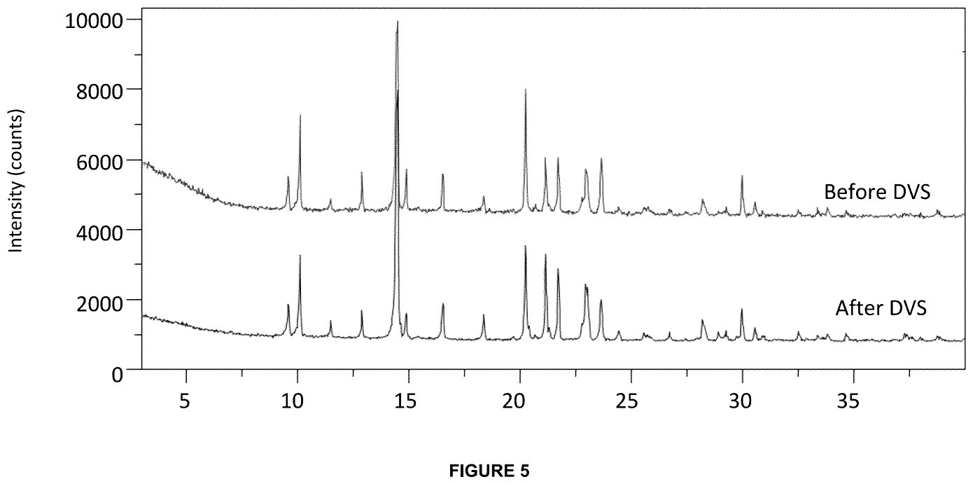

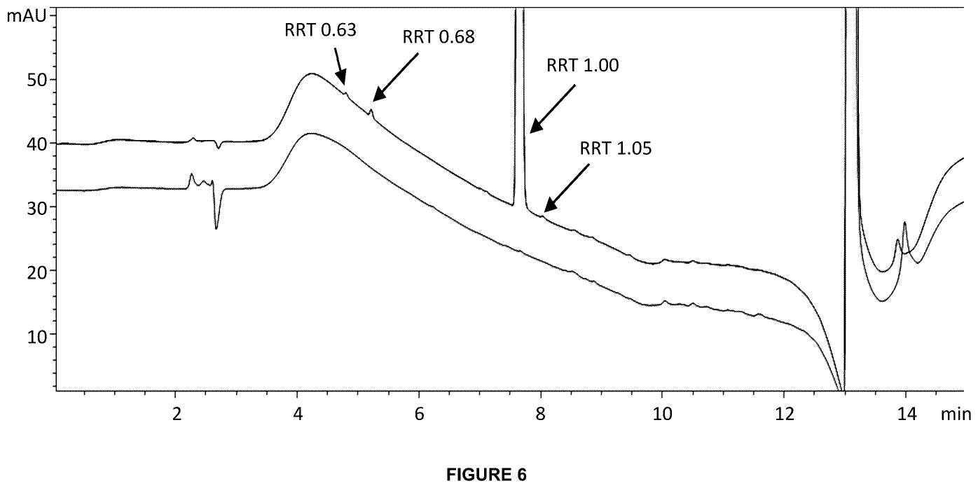

TGA and DSC data are shown in FIG. 2. A weight loss of 0.9% was observed up to 120° C. on the TGA curve.¹ The DSC result exhibited one sharp endotherm at 128.5° C. (onset temperature). Considering the low TGA weight loss and single sharp DSC endotherm, Form A was postulated to be an anhydrate. The PLM images shown in FIG. 3 indicated that irregular-shaped crystals with particle size of 50˜200 μm were observed. The DVS plot (FIG. 4) indicated that a water uptake of 0.024% was observed at 25° C./80% RH. XRPD overlay in FIG. 5 indicated that no form change was observed after DVS test. The HPLC purity of starting material was measured as 99.78 area % (see chromatogram of FIG. 6) and the impurity summary is listed in Table 2.

TABLE 2
Impurity summary of Compound (1) starting material

| #Peak | RRT | Area % |
|---|---|---|
| 1 | 0.63 | 0.05 |
| 2 | 0.68 | 0.12 |
| 3 | 1.00 | 99.78 |
| 4 | 1.05 | 0.06 |

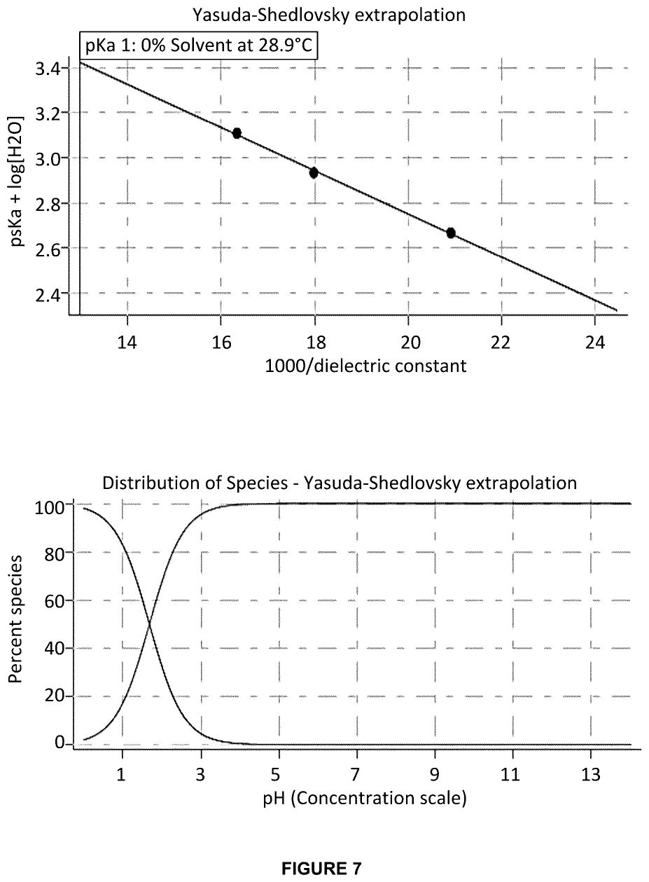

In addition, the pKa value of Compound (1) starting material was measured to be 1.68 by a Sirius pKa log P/D tester (model: T3) with a UV detector (UV metric method) using MeOH as solvent. The pKa value should be taken as reference because the effective pH range of UV metric method is pH 2-12. Detailed results of pKa measurement are listed in Table 3 and FIG. 7.

TABLE 3
pKa measurement results for Compound (1)

| Extrapolation | | | | | | Ionic | |
|---|---|---|---|---|---|---|---|
| type | pKa | % SD | Intercept | Slope | R₂ | strength | Temperature |
| Yasuda- | 1.68 | ±0.02 | 4.67 | −95.7995 | 0.9983 | 0.176M | 28.9° C. |
| Shedlovsky | | | | | | | |

¹ Description of the TGA data: The TGA value in the Figure shows a 0.9% weight loss. However we’ve prepared Form A with much lower volatile content (0.1% or lower).

### EXAMPLE 2—SOLID FORM SCREENING

A total of 96 solid form screening experiments were performed using different crystallization or solid transformation methods. The results are summarized in Table 4 and the experiment details are set forth below. Only one crystal form of Compound (1), Form A, was observed from screening.

TABLE 4
Summary of polymorph screening experiments

| Method | No. of Experiment | Result |
|---|---|---|
| Anti-solvent addition | 12 | Form A |
| Reverse anti-solvent addition | 8 | Form A |
| Slow evaporation | 13 | Form A |
| Slow cooling | 8 | Form A |
| Slurry at RT | 13 | Form A |
| Slurry at 50° C./70° C.* | 8 | Form A |
| Slurry Cycling (5~50° C.) | 10 | Form A |
| Vapor-solid diffusion | 8 | Form A |
| Vapor-solution diffusion | 8 | Form A |
| Polymer induced crystallization | 8 | Form A |
| Total | 96 | Form A |

*The slurry experiments were performed at 50° C. for 2 days, followed by slurrying at 70° C. for 3 days.

### Example 2.1—Anti-Solvent Addition

A total of 12 anti-solvent addition experiments were carried out. About 15 mg of Compound (1) starting material was dissolved in 0.1-0.5 mL solvent to obtain a clear solution and the solution was magnetically stirred (˜1000 rpm) followed by addition of 0.1 mL anti-solvent per step till precipitate appeared or the total amount of anti-solvent reached 10 mL. The obtained precipitate was isolated for XRPD analysis. Results, as summarized in Table 5, indicate that only Form A was generated.

TABLE 5
Summary of anti-solvent addition experiments

| Experiment ID | Solvent | Anti-solvent | Solid Form |
|---|---|---|---|
| 1* | MeOH | H₂O | Form A |
| 2* | Acetone | | Form A |
| 3* | THF | | Form A |
| 4* | 1,4-Dioxane | | Form A |
| 5 | DCM | n-Heptane | Form A |
| 6 | n-PrOAc | | Form A |
| 7 | MIBK | | Form A |
| 8 | CHCl₃ | Cyclohexane | Form A |
| 9 | MeOAc | | Form A |
| 10 | 2-MeTHF | | Form A |
| 11** | Dimethyl carbonate | m-Xylene | Form A |
| 12** | ACN | | Form A |

### Example 2.2—Reverse Anti-Solvent Addition

Reverse anti-solvent addition experiments were conducted under 8 conditions. Approximately 15 mg of Compound (1) starting material was dissolved in 0.1-0.3 mL of each solvent to get a clear solution. This solution was added dropwise into a glass vial containing 5 mL of each antisolvent at RT. The precipitate was isolated for XRPD analysis. Results, as summarized in Table 6, showed that only Form A was generated.

TABLE 6
Summary of reverse anti-solvent addition experiments

| Experiment # | Solvent | Anti-solvent | Solid Form |
|---|---|---|---|
| 1* | DMSO | H₂O | Form A |
| 2* | DMAc | | Form A |
| 3** | EtOAc | | Form A |
| 4 | CHCl3 | n-Heptane | Form A |
| 5 | IPAc | | Form A |
| 6 | DCM | Cyclohexane | Form A |
| 7 | Acetone | | Form A |
| 8** | NMP | m-Xylenes | Form A |

*Solid was obtained after stirring at 5° C.
**Clear solution was obtained after stirring at 5° C., and then transferred to RT for evaporation.

### Example 2.3 Slow Evaporation

Slow evaporation experiments were performed under 13 conditions. Briefly, ˜15 mg of Compound (1) starting material was dissolved in 0.2˜2.0 mL of solvent in a 3-mL glass vial. If not dissolved completely, suspensions were filtered using a PTFE membrane (pore size of 0.45 μm) and the filtrates would be used instead for the follow-up steps. The visually clear solutions were subjected to evaporation at RT with vials sealed by Parafilm® (poked with 6 pinholes). The solids were isolated for XRPD analysis, and the results, as summarized in Table 7, indicated that only Form A was obtained.

TABLE 7
Summary of slow evaporation experiments

| Experiment # | Solvent (v:v) | Solid Form |
|---|---|---|
| 1 | MeOH | Form A |
| 2 | Acetone | Form A |
| 3 | EtOAc | Form A |
| 4 | CPME | Form A |
| 5 | 2-MeTHF | Form A |
| 6 | ACN | Form A |
| 7 | DCM | Form A |
| 8 | 1,4-Dioxane | Form A |
| 9 | Dimethyl carbonate | Form A |
| 10 | THF | Form A |
| 11 | IPA | Form A |
| 12 | CHCl₃/MTBE (1:4) | Form A |
| 13 | MeOH/Toluene (1:4) | Form A |

### Example 2.4—Slow Cooling

Slow cooling experiments were conducted in 8 solvent systems. About 15 mg of Compound (1) starting material was suspended in 0.7 mL of solvent in an HPLC vial at RT. The suspension was then heated to 50° C., equilibrated for about 2 hours and filtered to a new vial using a PTFE membrane (pore size of 0.45 μm) if not completely dissolved. Filtrates were slowly cooled down to 5° C. at a rate of 0.1° C./min. The obtained solids were kept isothermal at 5° C. before isolated for XRPD analysis. Clear solutions were evaporated to dryness at RT and then solids were tested by XRPD. Results, summarized in Table 8, indicated Form A was obtained.

TABLE 8
Summary of slow cooling experiments

| | | Solid |
|---|---|---|
| Experiment # | Solvent (v:v) | Form |
| 1* | CPME | Form A |
| 2 | IPA | Form A |
| 3 | Toluene | Form A |
| 4* | EtOH | Form A |
| 5** | MTBE/Cyclohexane (1:1) | Form A |
| 6** | Acetone/n-Heptane (1:9) | Form A |
| 7 | EtOH/m-Xylene (1:1) | Form A |
| 8* | MeOH/H₂O (1:1) | Form A |

*Solid was obtained after stirring at 5° C.
**Clear solution was obtained after stirring at 5 C.° and −20° C., and then transferred to RT for evaporation.

### Example 2.5—Slurry at RT

Slurry conversion experiments were conducted at RT in 13 different solvent systems. ˜15 mg of Compound (1) starting material was suspended in 0.5 mL of solvent in an HPLC vial. After the suspension was stirred magnetically (˜700 rpm) for about 7 days at RT, the remaining solids were isolated for XRPD analysis. The results, as summarized in Table 9, showed that only Form A was generated.

TABLE 9
Summary of slurry conversion experiments at RT

| Experiment # | Solvent (v:v) | Solid Form |
|---|---|---|
| 1 | Cyclohexane | Form A |
| 2 | H₂O | Form A |
| 3 | n-Heptane | Form A |
| 4 | Toluene | Form A |
| 5 | CPME | Form A |
| 6 | MTBE | Form A |
| 7 | NMP/H₂O (1:9) | Form A |
| 8 | IPA/H₂O (0.97:0.03, a_{w}~0.3) | Form A |
| 9 | IPA/H₂O (0.92:0.08, a_{w}~0.6) | Form A |
| 10 | IPA/H₂O (0.77:0.23, a_{w}~0.9) | Form A |
| 11 | CHCl₃/m-Xylene (1:9) | Form A |
| 12 | MIBK/Cyclohexane (1:9) | Form A |
| 13 | IPAc/n-Heptane (1:9) | Form A |

### Example 2.6—Slurry at 50° C./70° C.

Slurry conversion experiments were also conducted at 50° C. in 8 different solvent systems. About 15 mg of Compound (1) starting material was suspended in 0.5 mL of solvent in an HPLC vial. After the suspension was magnetically stirred (˜700 rpm) for about 2 days at 50° C., the remaining solids were isolated for XRPD analysis and only Form A was generated. The samples were then transferred to stir at 70° C. for another 3 days, the remaining solids were isolated for XRPD analysis. Results, as summarized in Table 10, indicate that only Form A was generated.

TABLE 10
Summary of slurry conversion experiments at 50° C./70° C.

| Experiment | Solvent | Solid Form | Solid Form |
|---|---|---|---|
| # | (v:v) | (50° C.) | (70° C.) |
| 1 | H₂O | Form A | Form A |
| 2 | m-Xylene | Form A | Form A |
| 3* | Toluene | Form A | Form A |
| 4 | n-Heptane | Form A | Form A |
| 5 | ACN/H₂O (1:9) | Form A | Form A |
| 6 | IPA/Cyclohexane (1:9) | Form A | Form A |
| 7 | Anisole/n-Heptane (1:9) | Form A | Form A |
| 8* | EtOAc/m-Xylene (1:9) | Form A | Form A |

*Clear solution was obtained after 50° C. stirring, then ~20 mg starting material was further added.

### Example 2.7—Slurry Cycling (50-5° C.)

Slurry cycling (50-5° C.) experiments were conducted in 10 different solvent systems. About 15 mg of Compound (1) starting material was suspended in 0.5 mL of solvent in an HPLC vial. The suspensions were magnetically stirred (˜700 rpm) at 50° C. for 2 hours and then slowly cooled down to 5° C. at a rate of 0.1° C./min. The obtained solids were kept isothermal at 5° C. after cycled between 50° C. and 5° C. for 3 times. Solids were isolated for XRPD analysis. The results, as summarized in Table 11, indicate that only Form A was generated.

TABLE 11
Summary of slurry cycling (50-5° C.) experiments

| Experiment # | Solvent (v:v) | Solid Form |
|---|---|---|
| 1 | IPA | Form A |
| 2 | MTBE | Form A |
| 3 | Cyclohexane | Form A |
| 4 | CPME | Form A |
| 5 | Toluene | Form A |
| 6 | MeOH/H₂O (1:4) | Form A |
| 7 | Acetone/H₂O (1:4) | Form A |
| 8 | MTBE/n-Heptane (1:9) | Form A |
| 9 | Dimethyl carbonate/Cyclohexane | Form A |
| | (1:9) | |
| 10 | THF/m-Xylenes (1:9) | Form A |

### Example 2.8—Vapor Solid Diffusion

Eight vapor-solid diffusion experiments were performed using different solvents. About 15 mg of Compound (1) starting material was weighed into a 3-mL glass vial. This 3-mL vial was then placed into a 20-mL vial with 4 mL of solvents. The 20-mL vial was sealed with a cap and kept at RT for 7 days. The solids were isolated for XRPD analysis. The results, as summarized in Table 12, indicate that only Form A was generated.

TABLE 12
Summary of vapor-solid diffusion experiments

| Experiment # | Solvent | Solid Form |
|---|---|---|
| 1 | H₂O | Form A |
| 2 | EtOH | Form A |
| 3 | IPA | Form A |
| 4 | EtOAc | Form A |
| 5* | THF | Form A |
| 6 | 1,4-Dioxane | Form A |
| 7 | DMSO | Form A |
| 8 | Toluene | Form A |

*Clear solution was obtained, and then transferred to RT for evaporation.

### Example 2.9—Vapor-Solution Diffusion

Eight vapor-solution diffusion experiments were conducted. Approximate 15 mg of Compound (1) starting material was dissolved in 0.3-1.5 mL of appropriate solvent to obtain a clear solution in a 3-mL vial. This solution was then placed into a 20-mL vial with 4 mL of volatile solvents. The 20-mL vial was sealed with a cap and kept at RT allowing sufficient time for organic vapor to interact with the solution. Clear solution was obtained after 12 days and transferred to evaporate at RT. The solids were isolated for XRPD analysis. The results, as summarized in Table 13, indicate that only Form A was generated.

TABLE 13
Summary of vapor-solution diffusion experiments

| Experiment # | Solvent | Anti-solvent | Solid Form |
|---|---|---|---|
| 1 | THF | H₂O | Form A |
| 2 | ACN | | Form A |
| 3 | Acetone | | Form A |
| 4 | MeOAc | Cyclohexane | Form A |
| 5 | EtOH | | Form A |
| 6 | 2-MeTHF | n-Heptane | Form A |
| 7 | IPAc | | Form A |
| 8 | 1,4-Dioxane | m-Xylene | Form A |

### Example 2.10—Polymer Induced Crystallization

Polymer induced crystallization experiments were performed with two sets of polymer mixtures in 8 different solvent systems. Approximate 15 mg of Compound (1) starting material was dissolved in 0.5-1.5 mL of solvent in a 3-mL glass vial. About 1 mg of polymer mixture was added into the 3-mL glass vial. The resulting solutions were subjected to evaporation at RT with vials sealed by Parafilm® (poked with 3 pinholes) for slow evaporation. The solids were isolated for XRPD analysis. The results, as summarized in Table 14, indicate that only Form A was generated.

TABLE 14
Summary of polymer induced crystallization experiments

| Experiment ID | Solvent (v:v) | Polymer | Solid Form |
|---|---|---|---|
| 1 | IPA | Polymer | Form A |
| 2 | Toluene | mixture A | Form A |
| 3 | MeOAc | | Form A |
| 4 | n-PrOAc/EtOH (1:1) | | Form A |
| 5 | MTBE | Polymer | Form A |
| 6 | CHCl₃ | mixture B | Form A |
| 7 | Acetone | | Form A |
| 8 | MIBK/Toluene (1:1) | | Form A |

Polymer mixture A: polyvinyl pyrrolidone (PVP), polyvinyl alcohol (PVA), polyvinylchloride (PVC), polyvinyl acetate (PVAC), hypromellose (HPMC), methyl cellulose (MC) (mass ratio of 1:1:1:1:1:1). Polymer mixture B: polycaprolactone (PCL), polyethylene glycol (PEG), polymethyl methacrylate (PMMA) sodium alginate (SA), and hydroxyethyl cellulose (HEC) (mass ratio of 1:1:1:1:1).

### EXAMPLE 3—SINGLE CRYSTAL DATA FOR COMPOUND (1) FORM A

Block-like single crystals of Compound (1) Form A used for SCXRD characterization were crystallized from MeOH/toluene (1:4, v/v) solvent mixture by slow evaporation method. The experimental details are elaborated further below.

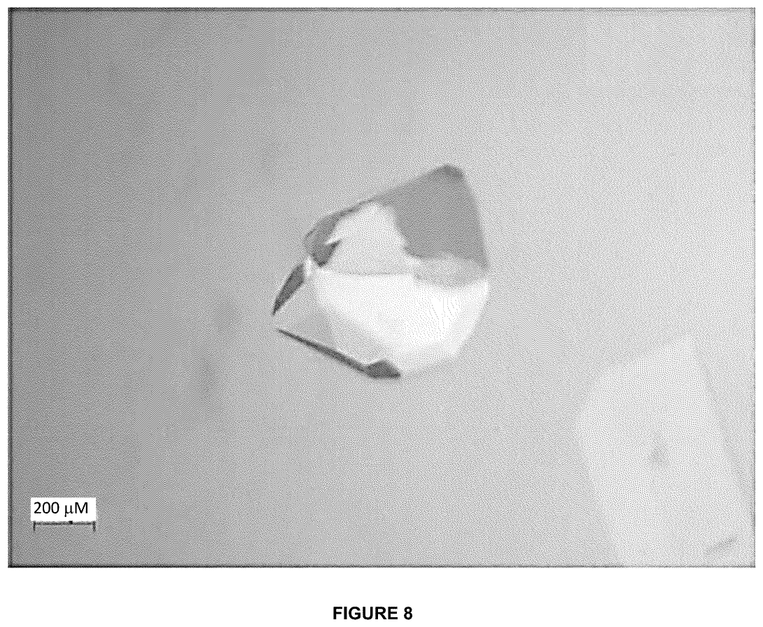

First, 14.7 mg of Compound (1) starting material was weighed into a 3-mL glass vial followed by addition of 1.5 mL MeOH/toluene (1:4, v/v) solvent mixture. After being oscillated on a vortex and ultrasonically shaken to accelerate dissolution, the suspension was then filtered through PTFE filter membrane (0.45 μm) and disposable syringe into a new 3-mL glass vial. The vial was then covered by seal membrane (Parafilm®) with six pinholes on it for slow evaporation at RT. After ˜10 days, block-like single crystals (CP ID: 814904-09-A13) were obtained as shown in FIG. 8.

A suitable single crystal with good diffraction quality was selected out from the block-like crystal samples and was wrapped with Paratone-N (an oil based cryoprotectant). The crystal was mounted on a mylar loop in a random orientation and immersed in a stream of nitrogen at 175 K. Preliminary examination and data collection were performed on a Bruker D8 VENTURE diffractometer (Mo/Kₐ radiation,) λ=0.71073 Å) and analyzed with the APEX3 software package.

Cell parameters and an orientation matrix for data collection were retrieved and refined (least-squares refinement) by SAINT (Bruker, V8.37A, after 2013) software using the setting angles of 9951 reflections in the range 2.333°<θ<27.040°. The data were collected to a maximum diffraction angle (θ) of 27.549° at 175K. The data set was 99.80% complete out to 27.549° in θ, having a Mean I/σ of 20.9 and D min (Mo) of 0.77 Å.

Frames were integrated with SAINT (Bruker, V8.37A, after 2013). A total of 36148 reflections were collected, of which 3204 were unique. Lorentz and polarization corrections were applied to the data. A multi-scan absorption correction was performed using SADABS-2014/5 (Bruker, 2014/5). wR₂(int) was 0.0981 before and 0.0709 after correction. The absorption coefficient μ of this material is 0.114 mm⁻¹ at this wavelength (λ=0.71073 Å) and the minimum and maximum transmissions are 0.7025 and 0.7456. Intensities of equivalent reflections were averaged. The agreement factor for the averaging was 6.17% based on intensity.

The structure was solved in the space group P2₁/c by Intrinsic Phasing using the ShelXT structure solution program, as set forth in Sheldrick, G. M. “A short history of SHELX,” Acta Crystallogr. Sect. A (2008) A64, 112-122, and refined by Least Squares using version 2017/1 of ShelXL (Sheldrick, Acta Crystallogr. (2015) C71, 3-8) refinement package contained in OLEX2 (Dolomanov et al. (2009), J. Appl. Cryst. 42, 339-341). All non-hydrogen atoms were refined anisotropically. The positions of hydrogen atoms were refined freely according to the Fourier Map.

The structure of the crystal was determined successfully. The crystal system is monoclinic and the space group is P2₁/c. The cell parameters are: a=9.3375(10) Å, b=8.5568(9) Å, c=17.6497(19) Å, α=90°, β=98.412(3)°, γ=90°, V=1395.0(3) Å³. The formula weight is 295.29 g·mol⁻¹ with Z=4, resulting in the calculated density of 1.406 g·cm⁻³. Further crystallographic data and the refinement parameters are listed in Table 18.

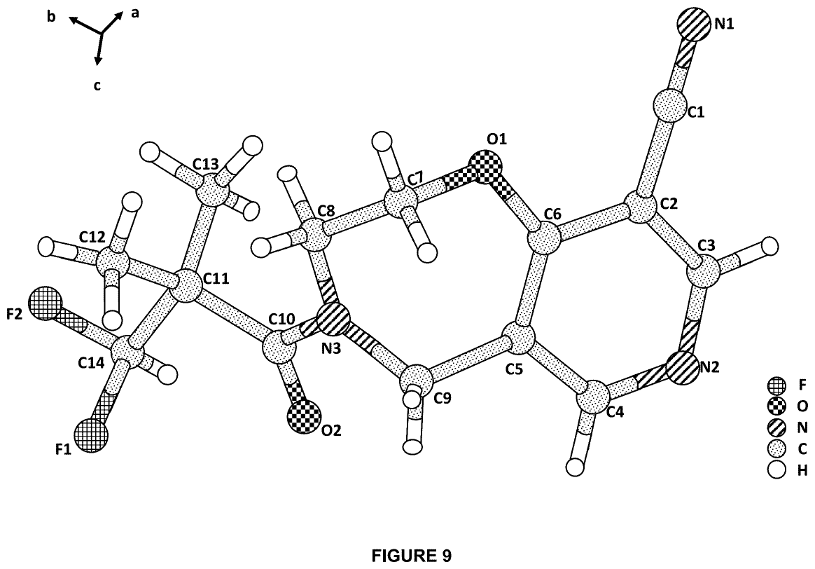

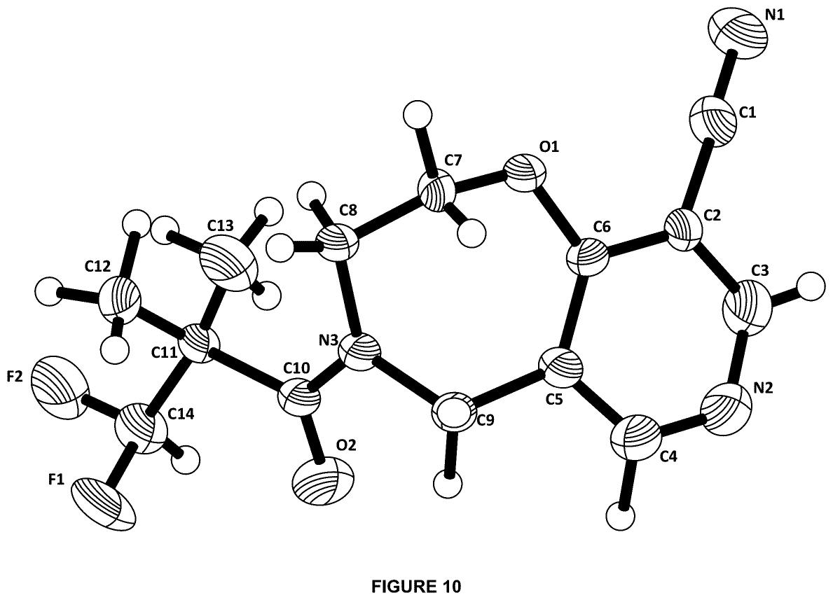

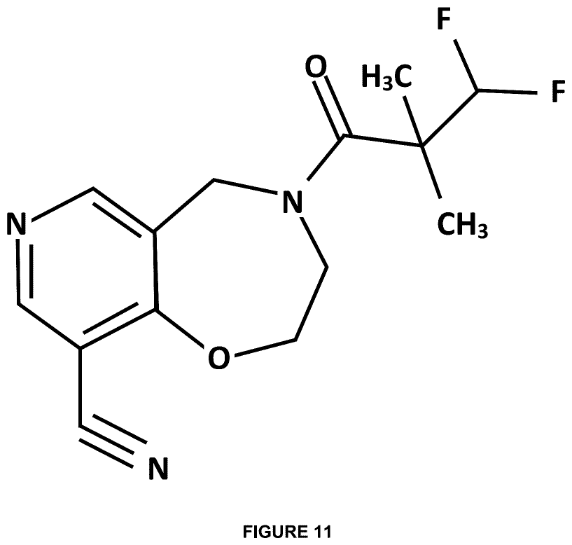

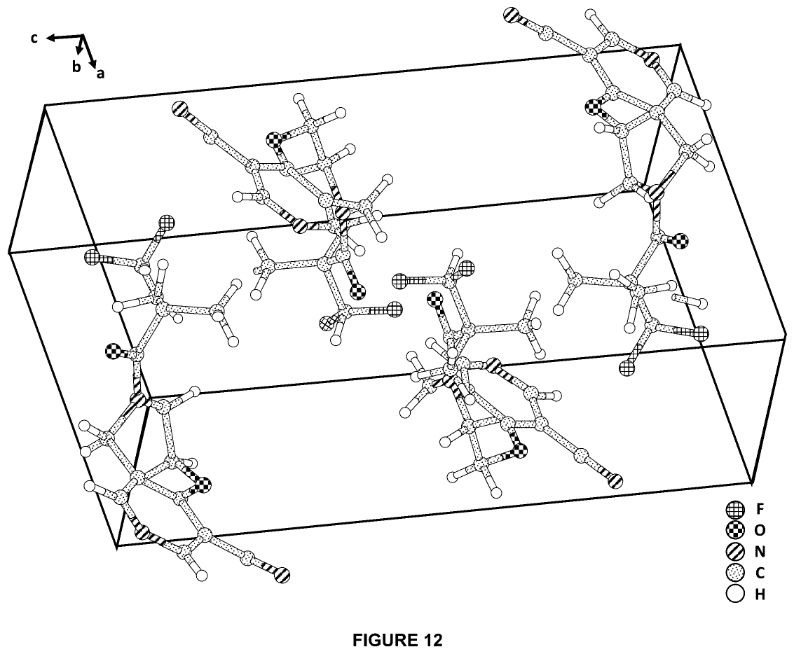

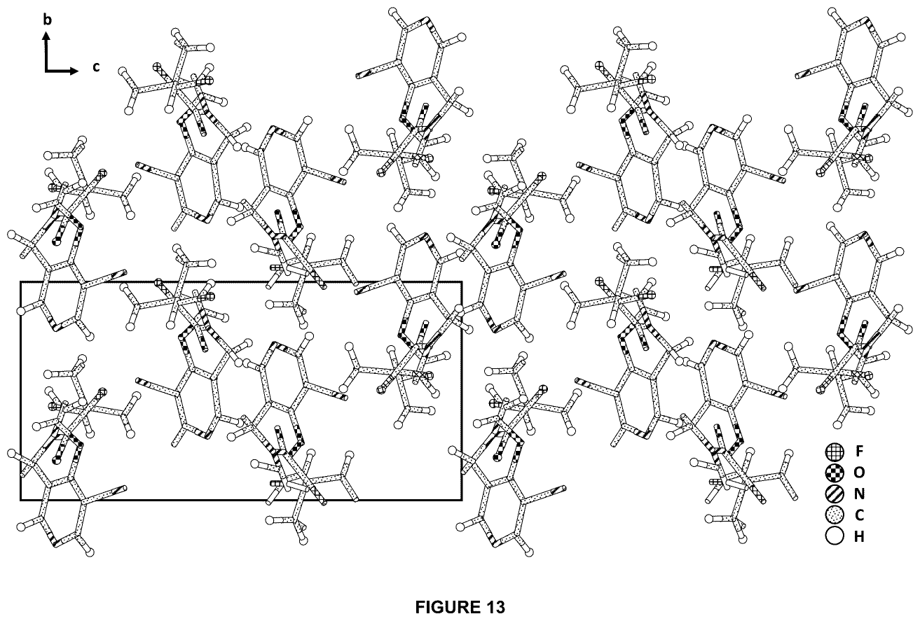

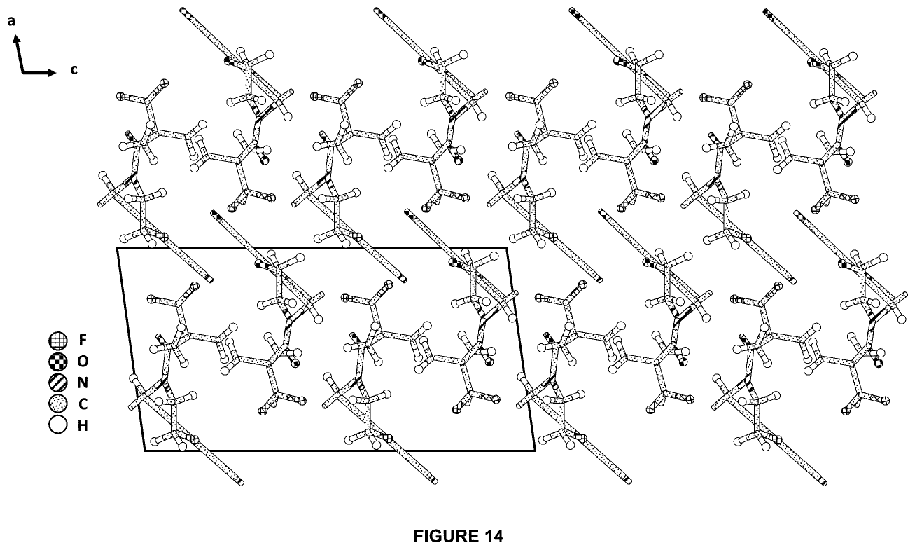

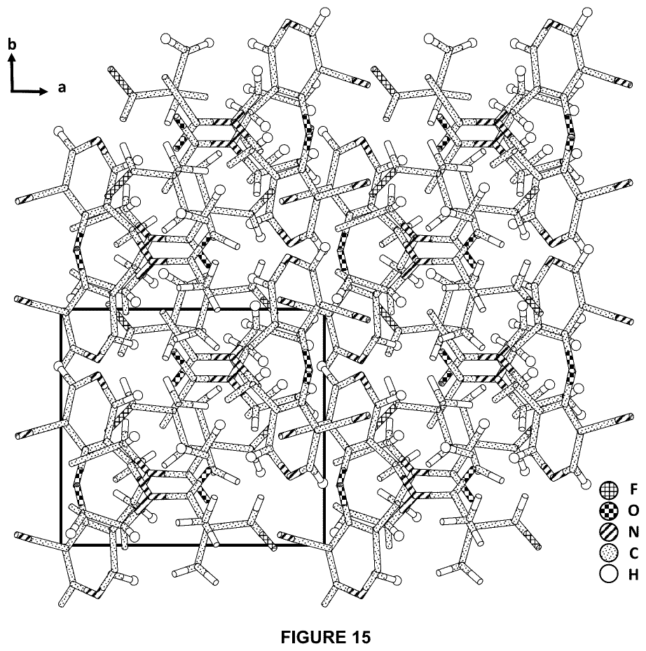

As shown in FIG. 9, the asymmetric unit of the single crystal structure is comprised of only one Compound (1) molecule, indicating the crystal is an anhydrate of Compound (1). The thermal ellipsoids drawing of the Compound (1) molecule in the crystal lattice is shown in FIG. 10. The single crystal structure determination confirmed that the structure of Compound (1) is consistent with the proposed chemical structure as shown in FIG. 11. The unit cell of the single crystal is shown in FIG. 12. The packing diagrams viewed along the crystallographic a-axis, b-axis, c-axis are shown in FIG. 13, FIG. 14, and FIG. 15, respectively.

The calculated XRPD pattern was generated for Cu radiation using Mercury⁴ program and the atomic coordinates, space group, and unit cell parameters from the single crystal structure. The calculated XRPD generated from the single crystal structure data and the experimental XRPD pattern of the single crystal sample are consistent with Compound (1) Form A reference as shown in Table 15.

TABLE 15
Crystallographic data and refinement parameters

| Identification code | Compound (1) Form A | |
|---|---|---|
| Empirical formula | C₁₄H₁₅F₂N₃O₂ | |
| Formula weight | 295.29 | |
| Temperature | 175 | K |
| Wavelength | Mo/Kα (λ = 0.71073 Å) | |
| Crystal system, space group | monoclinic, P2₁/c | |
| Unit cell dimensions | a = 9.3375(10) Å | |
| | b = 8.5568(9) Å | |
| | c = 17.6497(19) Å | |
| | α = 90° | |
| | β = 98.412(3)° | |
| | γ = 90° | |
| Volume | 1395.0(3) | Å³ |
| Z, Calculated density | 4, 1.406 g/cm³ | |
| Absorption coefficient | 0.114 | mm⁻¹ |
| F(000) | 616.0 | |
| Crystal size | 0.7 × 0.6 × 0.5 | mm³ |

| | |
|---|---|
| 2 Theta range for data collection | 5.934° to 55.098° |
| Limiting indices | −12 ≤ h ≤ 12 |
| | −11 ≤ k ≤ 11 |
| | −22 ≤ 1 ≤ 22 |
| Reflections collected/Independent | 36148/3204 [Rᵢₙₜ = 0.0617, |
| reflections | R_{sigma} = 0.0334] |
| Refinement method | Full-matrix least-squares on F² |
| Data/restraints/parameters | 3204/0/250 |
| Goodness-of-fit on F² | 1.045 |
| Final R indices [I ≥ 2sigma(I)] | R₁ = 0.0456, wR₂ = 0.1009 |
| Final R indices [all data] | R₁ = 0.0697, wR₂ = 0.1116 |
| Largest diff. peak and hole | 0.21/−0.25 e · Å⁻³ |

### EXAMPLE 4—COMPOUND (1) FORM A EVALUATION

### Example 4.1—Physical and Chemical Stability

To evaluate the physical and chemical stability, Compound (1) Form A was stored in 3 conditions (40° C./75% RH; 25° C./60% RH; and 60° C.) for one and four weeks. All samples were characterized using XRPD and HPLC purity, with the results summarized in Table 16.

TABLE 16
Stability evaluation summary of Form A

| Initial | Time | | | Final | Purity vs. |
|---|---|---|---|---|---|
| Form | point | Condition | Description | Form | initial (%) |
| Form | Initial | NA | White powder | Form A | NA |
| A | | 40° C./75% RH | White powder | Form A | 100.0 |
| | 1 week | 25° C./60% RH | White powder | Form A | 100.0 |
| | | 60° C. | White powder | Form A | 100.0 |
| | 4 weeks | 40° C./75% RH | White powder | Form A | 100.0 |
| | | 25° C./60% RH | White powder | Form A | 100.0 |
| | | 60° C. | White powder | Form A | 100.0 |

XRPD results from FIG. 17 to FIG. 18 indicated no form change was observed for Form A under all conditions. HPLC result indicated that no obvious HPLC purity change was observed. Detailed impurities of Form A were summarized in Table 17.

TABLE 17
Impurity summary of Form A after stability evaluation

| | | | % Area | |
|---|---|---|---|---|
| | | | Imp.1 (RRT | API (RRT |
| Initial Form | Time point | Condition | 0.68) | 1.00) |
| Form A | Initial | NA | 0.11 | 99.89 |
| | 1 week | 40° C./75% RH | 0.11 | 99.89 |
| | | 25° C./60% RH | 0.08 | 99.92 |
| | | 60° C. | 0.10 | 99.90 |
| | 4 weeks | 40° C./75% RH | 0.10 | 99.90 |
| | | 25° C./60% RH | 0.10 | 99.90 |
| | | 60° C. | 0.10 | 99.90 |

### Example 4.2—Kinetic Solubility

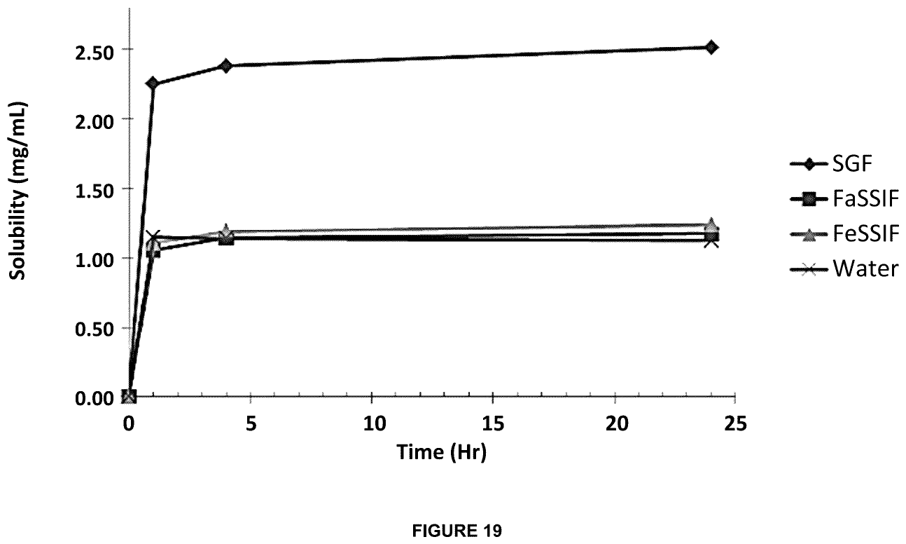

Kinetic solubility of Compound (1) Form A was evaluated in bio-relevant media (SGF, FaSSIF and FeSSIF) and H₂O at 37° C. for 1, 4, 24 hrs. Solids were suspended in FaSSIF, FeSSIF, SGF and H₂O with target conc. of ˜10 mg/mL. The suspensions were agitated on a rolling incubator at 25 rpm (in the incubator set at 37° C.) for 1, 4 and 24 hrs. At each time point, 1 mL of the suspension was pipetted out for centrifugation at 15000 rpm (3 min) and filtration through 0.45 μm membrane to obtain supernatant for HPLC solubility and pH tests, the residual solids were analyzed by XRPD. The solubility data of Form A are summarized in Table 18 and the solubility curves are shown in FIG. 19.

TABLE 18
Summary of kinetic solubility results of Form A

| | | Time | | | | |
|---|---|---|---|---|---|---|
| Initial | | point | Final | Solubility | Obser- | Final |
| Form | Media | (hr) | Form | (mg/mL) | vation | pH |
| Form A | SGF | 1 | Form A | 2.3 | Turbid | 1.8 |
| | (pH 1.8) | 4 | Form A | 2.4 | Turbid | 2.3 |
| | | 24 | Form A | 2.5 | Turbid | 2.2 |
| | FaSSIF | 1 | Form A | 1.1 | Turbid | 6.4 |
| | (pH 6.5) | 4 | Form A | 1.1 | Turbid | 6.6 |
| | | 24 | Form A | 1.2 | Turbid | 6.6 |
| | FeSSIF | 1 | Form A | 1.1 | Turbid | 5.6 |
| | (pH 5.0) | 4 | Form A | 1.2 | Turbid | 5.6 |
| | | 24 | Form A | 1.2 | Turbid | 5.6 |
| | H₂O | 1 | Form A | 1.1 | Turbid | 8.5 |
| | (pH 6.5) | 4 | Form A | 1.1 | Turbid | 8.4 |
| | | 24 | Form A | 1.1 | Turbid | 8.7 |

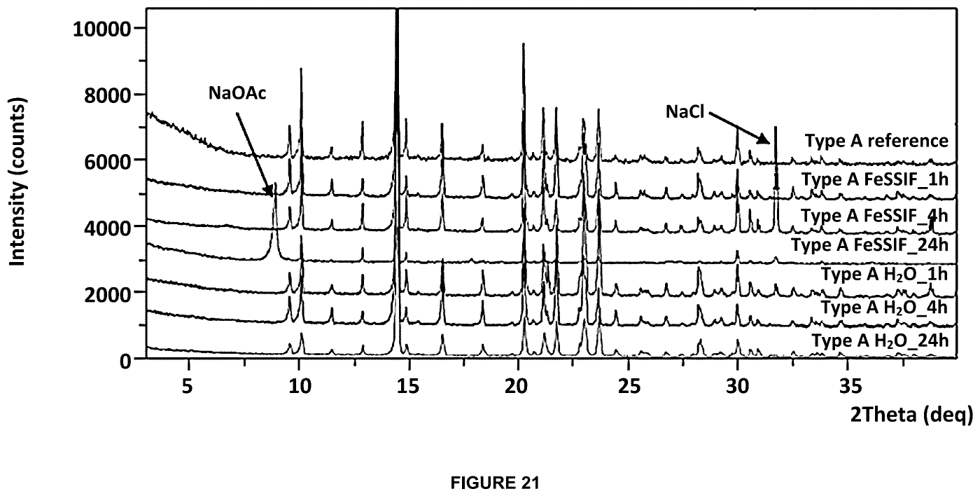

No form change was observed after kinetic solubility test in bio-relevant media or H₂O. The XRPD overlays are displayed in FIG. 20 and FIG. 21.

### Example 4.3—pH Solubility

24-Hrs solubility of Form A was measured in pH buffers (i.e., pH 2.0, 4.0, 6.0, 7.0, 8.0) at RT. Solids were suspended in pH buffers with target conc. of ˜10 mg/mL. The suspensions were stirred (1000 rpm) at 37° C. for 24 hrs, prior to centrifugation at 12000 rpm (2 min) and filtration through 0.45 μm membrane to obtain supernatant for HPLC solubility and pH tests, the residual solids were analyzed by XRPD. Detailed results were summarized in Table 19.

TABLE 19
24-Hrs solubility results summary of Form A in pH buffers

| Experi- | | Final | Solubility | Observa- | Final |
|---|---|---|---|---|---|
| ment # | Media | form | (mg/mL) | tion | pH |
| 1 | pH 2.0 | Form A | 1.8 | Turbid | 2.3 |
| | (50 mM HCl—KCl) | | | | |
| 2 | pH 4.0 | Form A | 0.84 | Turbid | 4.1 |
| | (50 mM Citrate) | | | | |
| 3 | pH 6.0 | Form A | 0.75 | Turbid | 5.9 |
| | (50 mM Citrate) | | | | |
| 4 | pH 7.0 | Form A | 0.90 | Turbid | 6.9 |
| | (50 mM Phosphate) | | | | |
| 5 | pH 8.0 | Form A | 0.81 | Turbid | 7.8 |
| | (50 mM Phosphate) | | | | |

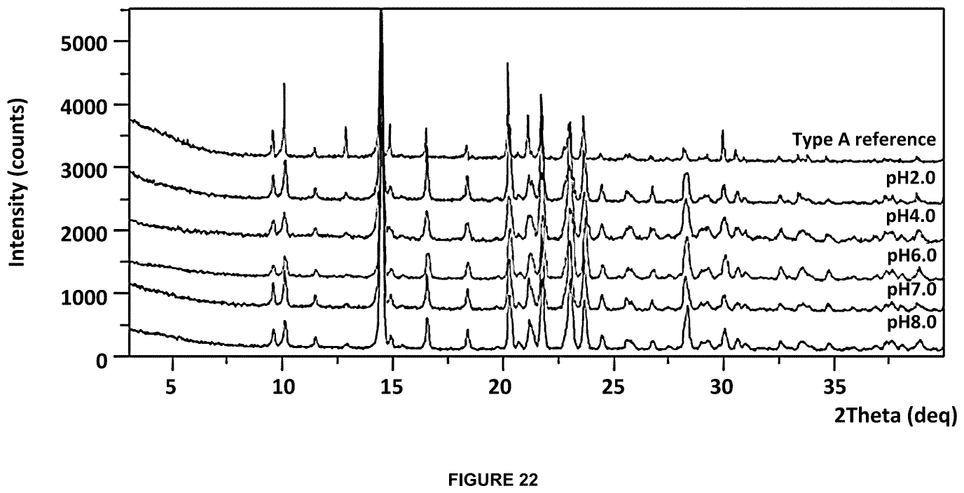

As shown in FIG. 22, no form change was observed for Form A after equilibrium solubility evaluation in pH buffers.

### Example 4.4—Solution Stability Evaluation

Solution stability study was performed in pH 2.0/4.0/6.0/7.0 (24 hrs) and pH 8.0 (24 hrs and 96 hrs) buffers. Solids were dissolved with pH buffers with target conc. of ˜0.5 mg/ml to form clear solutions and stored at 37° C. for 24 hrs or 96 hrs. The stability results are summarized in Table 20 and Table 21.

TABLE 20
Summary of solution stability results in pH buffers

| Experiment | | Time | | Purity vs. |
|---|---|---|---|---|
| # | Media | point | Observation | Initial (%) |
| 1 | pH 2.0 | 24 hrs | Clear | 85.1 |
| | (50 mM HCl—KCl) | | | |
| 2 | pH 4.0 | 24 hrs | Clear | 98.5 |
| | (50 mM Citrate) | | | |
| 3 | pH 6.0 | 24 hrs | Clear | 99.8 |
| | (50 mM Citrate) | | | |
| 4 | pH 7.0 | 24 hrs | Clear | 99.9 |
| | (50 mM Phosphate) | | | |
| 5 | pH 8.0 | 24 hrs | Clear | 100.0 |
| | (50 mM Phosphate) | | | |
| 6 | pH 8.0 | 96 hrs | Clear | 100.0 |
| | (50 mM Phosphate) | | | |

TABLE 21
Impurity summary of solution stability in pH buffers

| | | | % Area | | | | |
|---|---|---|---|---|---|---|---|
| | | | Imp. | Imp. | Imp. | | Imp. |
| | | | 1 | 1 | 1 | API | 1 |
| Experi- | | Time | (RRT | (RRT | (RRT | (RRT | (RRT |
| ment # | Media | point | 0.66) | 0.70) | 0.76) | 1.00) | 1.05) |
| 1 | pH 2.0 (50 | 24 hrs | 0.15 | 0.67 | 14.25 | 84.93 | — |
| | mM HCl—KCl) | | | | | | |
| 2 | pH 4.0 (50 | 24 hrs | 0.15 | — | 0.38 | 98.42 | 0.06 |
| | mM Citrate) | | | | | | |
| 3 | pH 6.0 (50 | 24 hrs | 0.13 | — | 0.06 | 99.75 | 0.06 |
| | mM Citrate) | | | | | | |
| 4 | pH 7.0 (50 | 24 hrs | 0.15 | — | — | 99.78 | 0.07 |
| | mM Phosphate) | | | | | | |
| 5 | pH 8.0 (50 | 24 hrs | 0.13 | — | — | 99.81 | 0.06 |
| | mM Phosphate) | | | | | | |
| 6 | pH 8.0 (50 | 96 hrs | 0.13 | — | — | 99.83 | 0.04 |
| | mM Phosphate) | | | | | | |

—: <0.05 area %

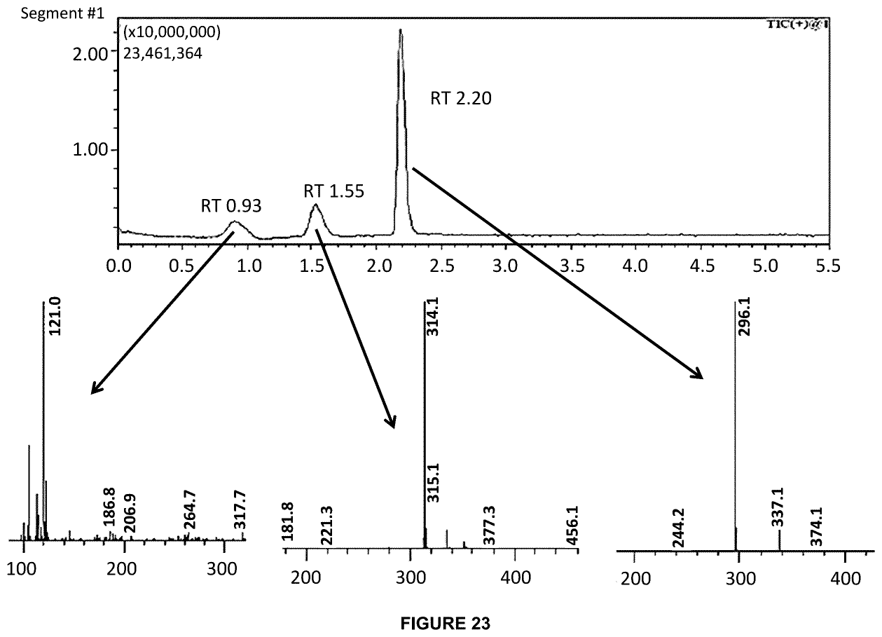

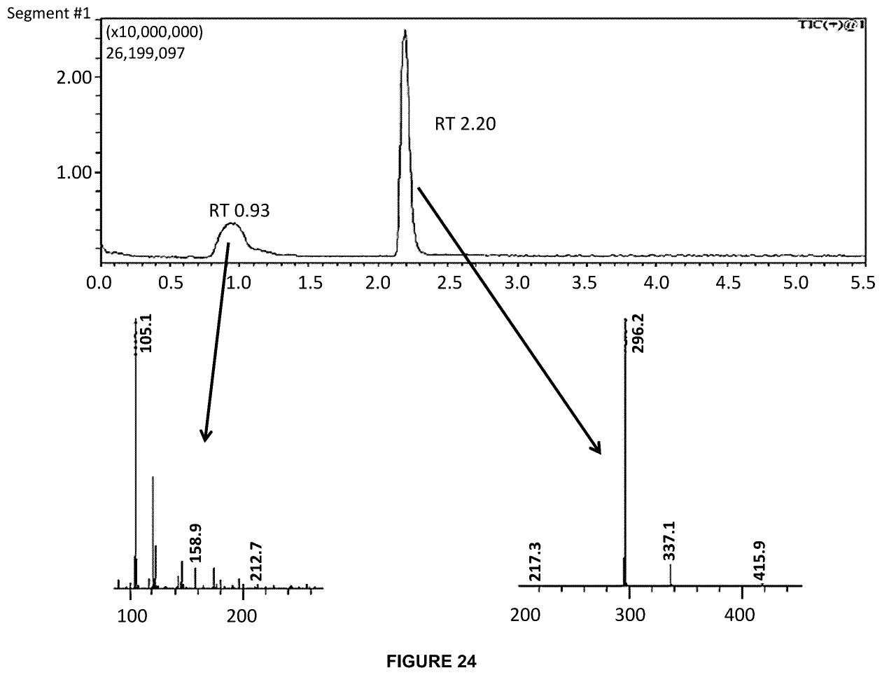

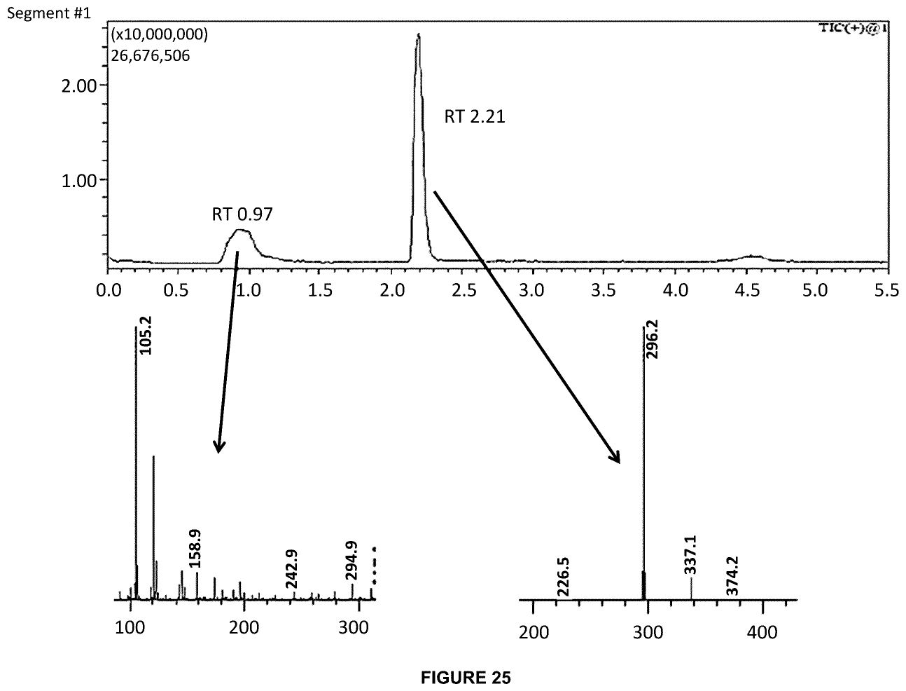

Degradation was observed in pH 2.0 and pH 4.0 buffers. For pH 2.0 and pH 8.0 samples, LC-MS was performed to determine the molecular weight of the impurities. The LC chromatograms and mass spectra are shown in FIGS. 23-25.

### Example 5—In Silico Polymorphism Study of Compound (I)

### Hardware

The calculations were carried out on 384 cores of Intel XEON ES processors or equivalent hardware.

### COMPUTATIONAL DETAILS

### Description of the Compound

Compound (I) contains five flexible torsion angles, including two methyl groups, and one flexible ring. The compound contains no chiral centers.

### Standard Search Space (for Possible Deviations, See Below)

Crystal structures were first generated with one (Z′=1) molecule per asymmetric unit. According to the statistics of the Cambridge Structural Database (CSD), 88.3% of all compounds crystallize with one molecule per asymmetric unit.

The crystal structure generation was carried out in 38 space groups that cover 99.92% of the crystal structures with Z′=1 according to CSD statistics (P1, P-1, P2₁, C2, Pc, Cc, P2/c, P2₁/c, C2/c, P2₁2₁2, P2₁2₁2₁, C222₁, Pca2₁, Pna2₁, Aba2, Fdd2, 1ba2, Pcca, Pccn, Pbcn, Pbca, Fddd, P4₁, I4, I4₁, I-4, P42/n, I4₁/a, P4₁2₁2, I4₁cd, P-42₁c, P3₁, R3, R-3, P3₁21, R3c, P6₁, P6₁22). A value of Δ=1.0 kcal/mol was chosen for the target energy window in which the completeness of the CSP procedure is statistically controlled.

After the Z′=1 structure generation, crystal structures with two molecules per asymmetric unit were constructed from Z′=1 structures by, e.g., unit-cell doubling. This stage is known as the smart Z′=2 CSP.

Crystal structures were also generated with two molecules per asymmetric unit (Z′=2) in a standard search. According to the statistics of the Cambridge Structural Database (CSD), 10.5% of all compounds crystallize with two molecules per asymmetric unit. The Z′=2 case was dealt with in two independent rounds. In the first round, only the space groups P1, P-1 and P2₁ were considered which cover 42.5% of the Z′=2 cases in the CSD. In the second round, the space groups C2, Pc, Cc, P2₁/c, C2/c, P2₁2₁2₁, Pca2₁, Pna2₁ were considered. These space groups cover an additional 53.4% of the Z′=2 cases.

### Deviations From the Standard Procedure

None.

### CPU Time Consumption

The tailor-made force field was generated in 4 days. The actual crystal structure prediction took 60 days.

The PBE(0)+MBD energy calculations for 216 structures took 4 days.

The PBE(0)+MBD+Fvib energy calculations for 5 structures took 2.5 days.

TABLE 22
Some numbers from the energy calculations

| | | | Z′ = 2 | Z′ = 2 |
|---|---|---|---|---|
| | Z′ = 1 | Smart Z′ = 2 | part I | part II |
| Step 1 | 10,000 | 6538 | 702 | 7825 |
| Step 2 | 194 | 1465 | 179 | 1670 |
| Step 3 | 26 | 161 | 15 | 15 |
| σ(Step 1 → Step 2)¹ | 0.17 | 0.16 | 0.20 | 0.16 |
| σ(Step 2 → Step 3)¹ | 0.003 | 0.005 | 0.002 | 0.002 |
| Step 1 convergence | 99% | 100% | 95% | 95% |
| Step 1 energy window | 4.6σ | 3.8σ | 2.9σ | 2.9σ |

¹kcal/mol/\sqrt{Nₐₜₒₘₛ}

In step 4, the energies of all 216 step 3 structures were computed with PBE(0)+MBD.

### Predicted Structures

The full list of OFT-optimized crystal structures with step 4 (free) energies calculated at the PBE(0)+MBD (+Fvib) level is supplied as supplementary materials in the archive file results.zip. The file “table.txt” contains text information about all 216 step 4 crystal structures. The actual crystal structures are contained in the “structures” subdirectory.

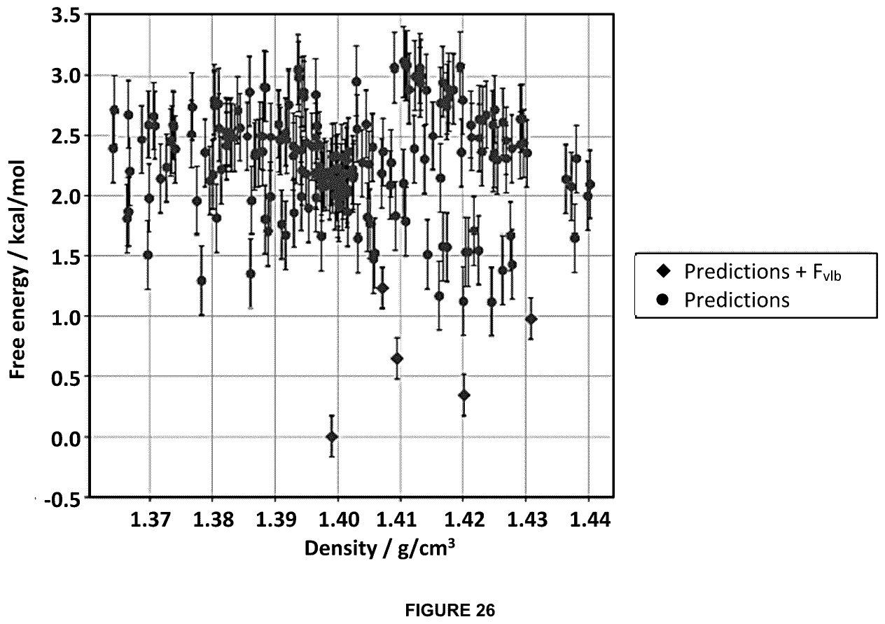

Table 23 lists the 30 most stable predicted crystal structures and FIG. 26 shows the free energy landscape.

TABLE 23
Some properties of the 30 most stable predicted structures
from step 4, with F_{vib} correction computed at 298.15K

| | | Exergy | | | | | | | | | |
|---|---|---|---|---|---|---|---|---|---|---|---|
| | Energy | error | Density | | Space | a | b | c | α | β | γ |
| Rank | [kcal/mol] | [kcal/mol] | [g/cm³] | Z' | group | [Å] | [Å] | [Å] | [°] | [°] | [°] |
| 1 | 0.000 | 0.172 | 1.399 | 1 | P2₁/c | 9.339 | 8.604 | 17.663 | 90 | 98.995 | 90 |
| 2 | 0.343 | 0.172 | 1.420 | 1 | P-1 | 8.534 | 9.062 | 9.415 | 99.347 | 85.089 | 105.825 |
| 3 | 0.647 | 0.172 | 1.409 | 2 | P2₁/c | 9.320 | 8.622 | 35.152 | 90 | 80.143 | 90 |
| 4 | 0.977 | 0.172 | 1.431 | 1 | P2₁/c | 9.450 | 14.734 | 9.882 | 90 | 85.048 | 90 |
| 5 | 1.118 | 0.285 | 1.425 | 1 | P2₁/c | 10.558 | 7.211 | 18.987 | 90 | 107.763 | 90 |
| 6 | 1.123 | 0.285 | 1.420 | 1 | P3₁ | 10.894 | 10.894 | 10.078 | 90 | 90 | 120 |
| 7 | 1.165 | 0.285 | 1.416 | 1 | C2/c | 11.538 | 12.466 | 22.041 | 90 | 60.889 | 90 |
| 8 | 1.231 | 0.172 | 1.407 | 1 | P2₁/c | 9.748 | 10.799 | 13.727 | 90 | 105.282 | 90 |
| 9 | 1.293 | 0.285 | 1.378 | 2 | P-1 | 7.172 | 14.124 | 15.298 | 112.655 | 93.468 | 84.496 |
| 10 | 1.351 | 0.285 | 1.386 | 2 | P2₁/c | 8.589 | 35.111 | 9.387 | 90 | 88.683 | 90 |
| 11 | 1.378 | 0.285 | 1.426 | 1 | Pbca | 11.824 | 9.546 | 24.367 | 90 | 90 | 90 |
| 12 | 1.427 | 0.285 | 1.428 | 2 | Pna2₃ | 11.750 | 32.136 | 7.276 | 90 | 90 | 90 |
| 13 | 1.473 | 0.285 | 1.406 | 1 | P2₁/c | 18.973 | 6.089 | 12.303 | 90 | 79.020 | 90 |
| 14 | 1.505 | 0.285 | 1.370 | 2 | P2₁/c | 15.323 | 7.315 | 27.856 | 90 | 113.471 | 90 |
| 15 | 1.509 | 0.285 | 1.414 | 1 | C2/c | 22.125 | 6.380 | 22.687 | 90 | 119.991 | 90 |
| 16 | 1.520 | 0.285 | 1.406 | 2 | P2₁/c | 11.045 | 20.639 | 16.494 | 90 | 47.906 | 90 |
| 17 | 1.529 | 0.285 | 1.420 | 1 | P2₁/c | 9.511 | 15.871 | 11.308 | 90 | 53.985 | 90 |
| 18 | 1.530 | 0.285 | 1.421 | 2 | C2/c | 13.891 | 10.470 | 38.428 | 90 | 81.089 | 90 |
| 19 | 1.544 | 0.285 | 1.423 | 2 | C2/c | 11.083 | 13.153 | 39.339 | 90 | 74.101 | 90 |
| 20 | 1.569 | 0.285 | 1.417 | 2 | P-1 | 8.800 | 13.872 | 13.670 | 57.463 | 91.465 | 82.138 |
| 21 | 1.576 | 0.285 | 1.417 | 2 | P2₁/c | 13.650 | 8.800 | 23.097 | 90 | 86.369 | 90 |
| 22 | 1.641 | 0.285 | 1.403 | 1 | Pccn | 11.912 | 24.645 | 9.522 | 90 | 90 | 90 |
| 23 | 1.645 | 0.285 | 1.438 | 1 | P2₁/c | 6.001 | 13.119 | 17.447 | 90 | 83.262 | 90 |
| 24 | 1.660 | 0.285 | 1.397 | 2 | P2₁/c | 8.577 | 34.857 | 9.426 | 90 | 95.026 | 90 |
| 25 | 1.662 | 0.285 | 1.428 | 2 | P-1 | 8.956 | 11.982 | 14.951 | 67.067 | 96.017 | 73.495 |
| 26 | 1.669 | 0.285 | 1.392 | 2 | P2₁/c | 8.468 | 35.412 | 9.432 | 90 | 94.763 | 90 |
| 27 | 1.698 | 0.285 | 1.389 | 1 | P2₁/c | 5.904 | 17.558 | 14.590 | 90 | 110.969 | 90 |
| 28 | 1.704 | 0.285 | 1.422 | 1 | P-1 | 6.385 | 8.963 | 12.136 | 92.028 | 95.987 | 87.474 |
| 24 | 1.756 | 0.285 | 1.391 | 2 | P-1 | 8.655 | 11.689 | 15.765 | 67.129 | 80.189 | 98.083 |
| 30 | 1.764 | 0.285 | 1.405 | 2 | P-1 | 8.639 | 13.100 | 13.307 | 110.443 | 97.121 | 91.760 |

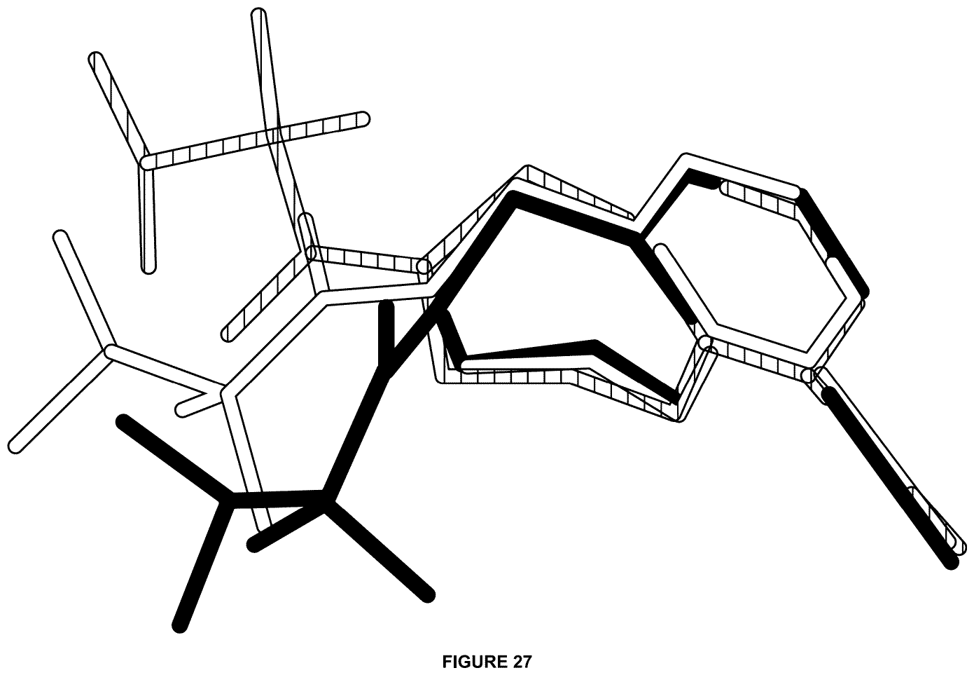

There are no voids greater than 20 Å³/Z in any of the predicted structures. The compound contains no hydrogen-bond donors. Although the molecule is fairly rigid, its shape can change considerably between crystal structures, as shown in FIG. 27.

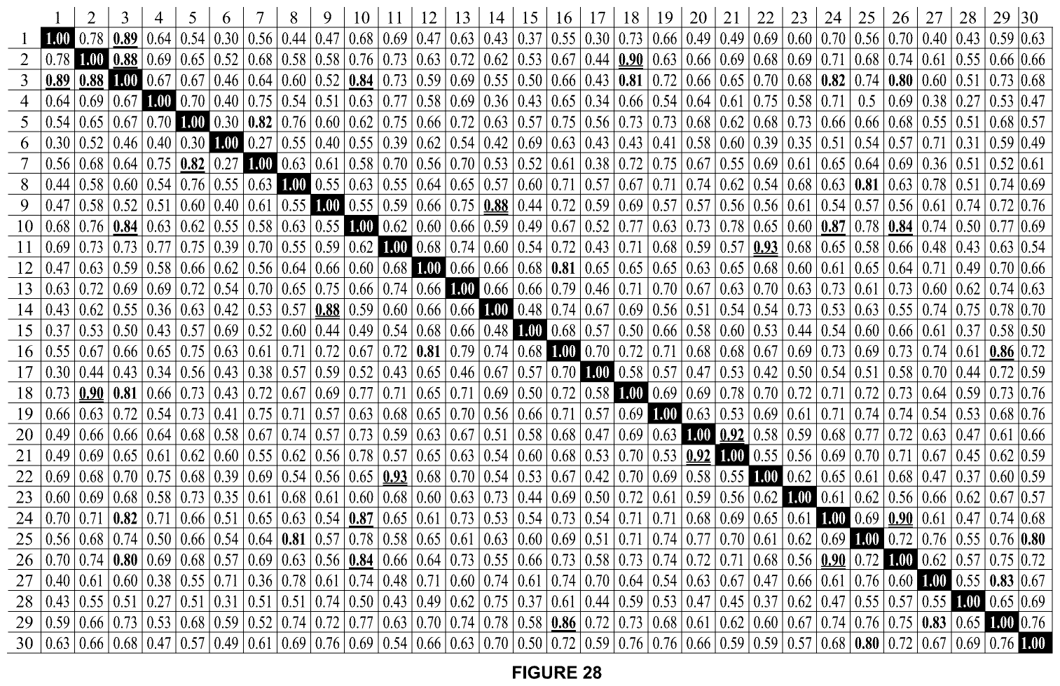

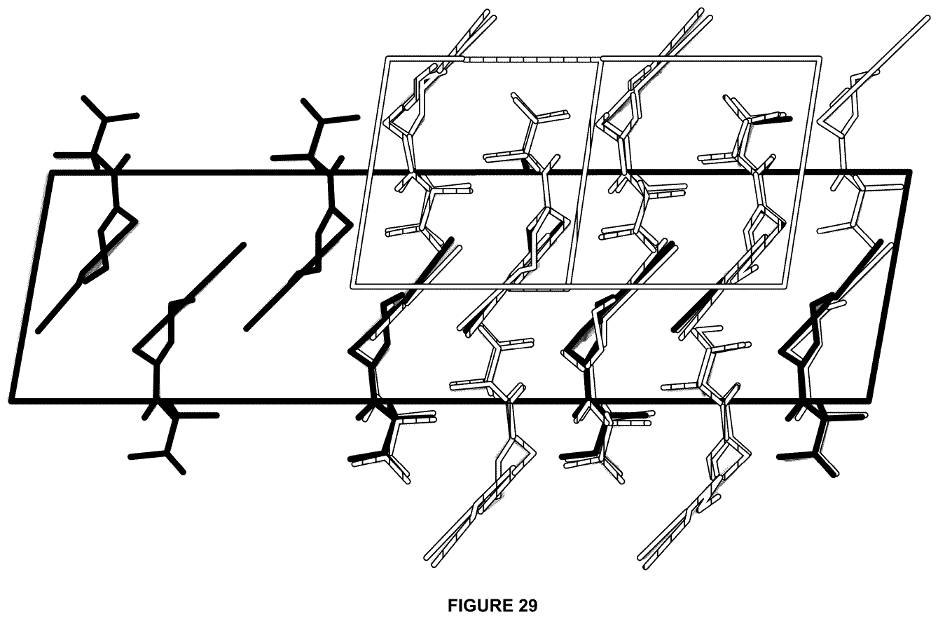

A similarity matrix was calculated for the first 30 structures as the normalized cross-correlation between the simulated powder diffraction patterns. This is graphically represented in FIG. 28 in which the similarity matrix is shown with values from 0.8 to 1.0 colored on a white-green color scale. Ranks 1, 2 and 3 show some similarity; indeed in projection they can be overlaid (see FIG. 29). In three dimensions, ranks 1, 2 and 3 are similar but different.

### Comparison With Compound (I), Form A Single-Crystal Data

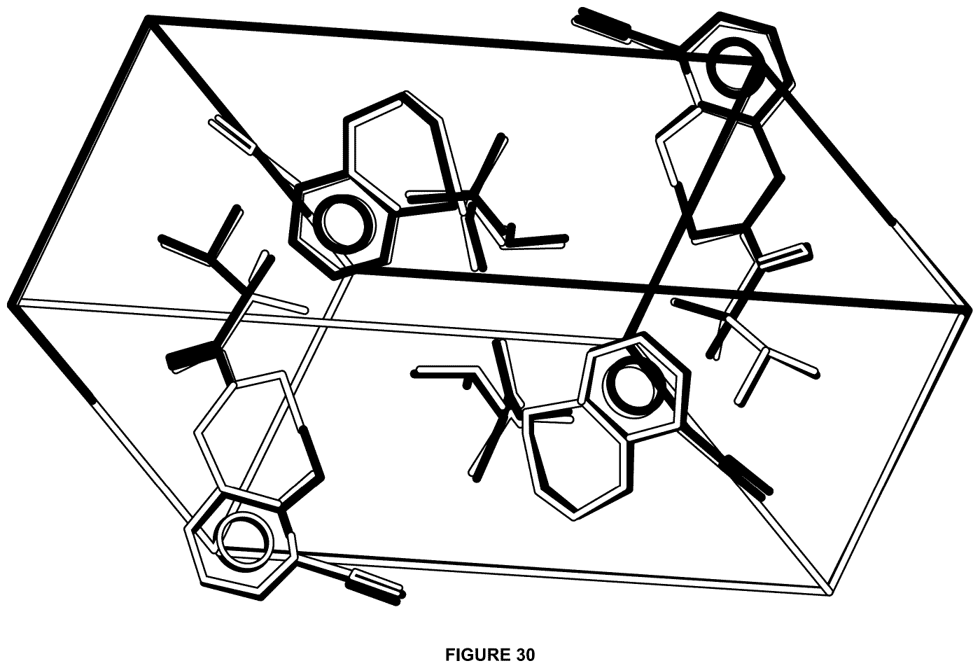

Form A matches the predicted rank 1 structure. FIG. 30 shows the overlay of form A with rank 1.

### Free Energy Landscape in the Context of the Experimental Structures

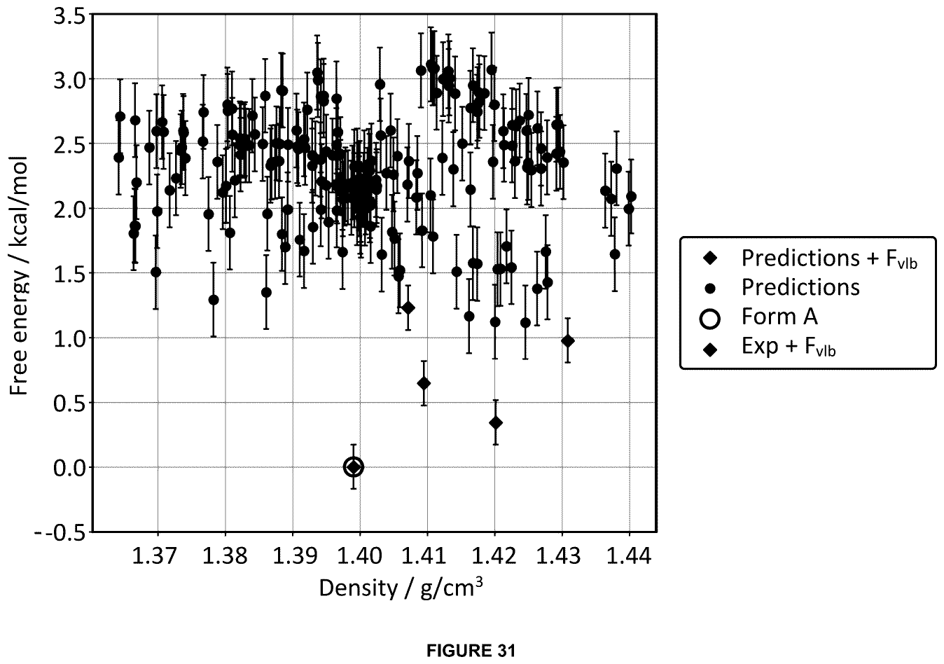

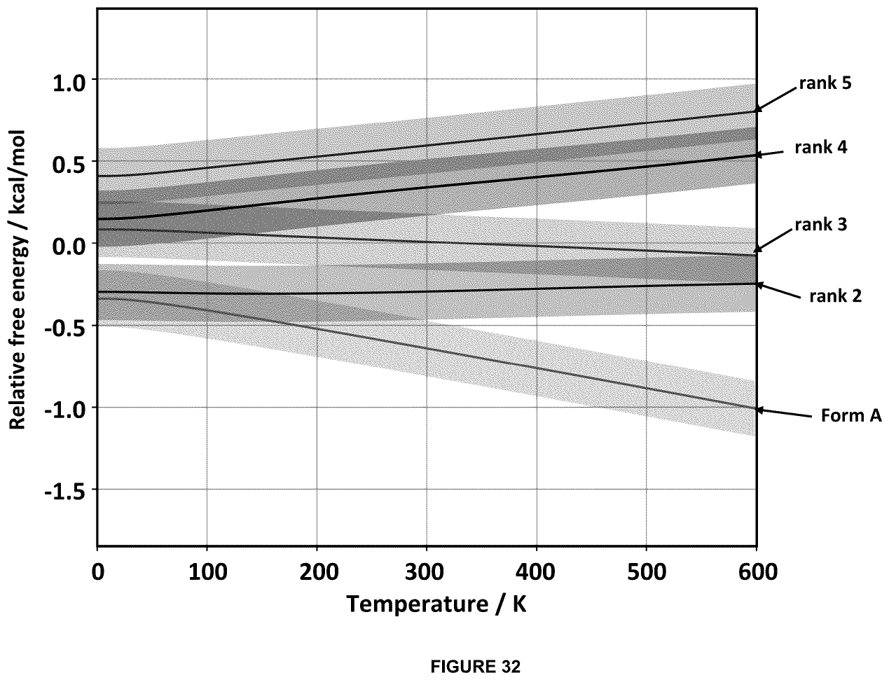

As seen in FIGS. 31-32, the most stable predicted structure (rank 1) matches Form A. Ranks 1, 2 and 3 are very similar, and from a kinetics point of view, if one of these could crystallize then all of them could crystallize. We therefore interpret the fact that rank 1 (=Form A) crystallized as meaning that rank 1 (=Form A) is the thermodynamically most stable structure; this is in agreement with the calculations. The first rank that is not similar to Form A is rank 4, 0.977 kcal/mol less stable than Form A. The error bar is 0.172 kcal/mol, so rank 4 is more than 50 away from Form A.

### EXAMPLE 5—AMORPHOUS FORMS

### Example 5.1 Purely Amorphous Form

A purely amorphous form of 4-(3,3-difluoro-2,2-dimethylpropanoyl)-2,3,4,5-tetrahydropyrido[3,4-f][1,4]oxazepine-9-carbonitrile was made by placing a small sample of the compound into a 2 mL glass vial and heating it at 135° C. for about 1 min until the compound melts to an oil. Thereafter the vial was flash cooled in a dry-ice acetone bath, and the resulting product was immediately (within 5 minutes) analyzed by XRPD as described herein.

### Example 5.2 Substantially Amorphous Form

A substantially amorphous form of 4-(3,3-difluoro-2,2-dimethylpropanoyl)-2,3,4,5-tetrahydropyrido[3,4-f][1,4]oxazepine-9-carbonitrile was made as follows:

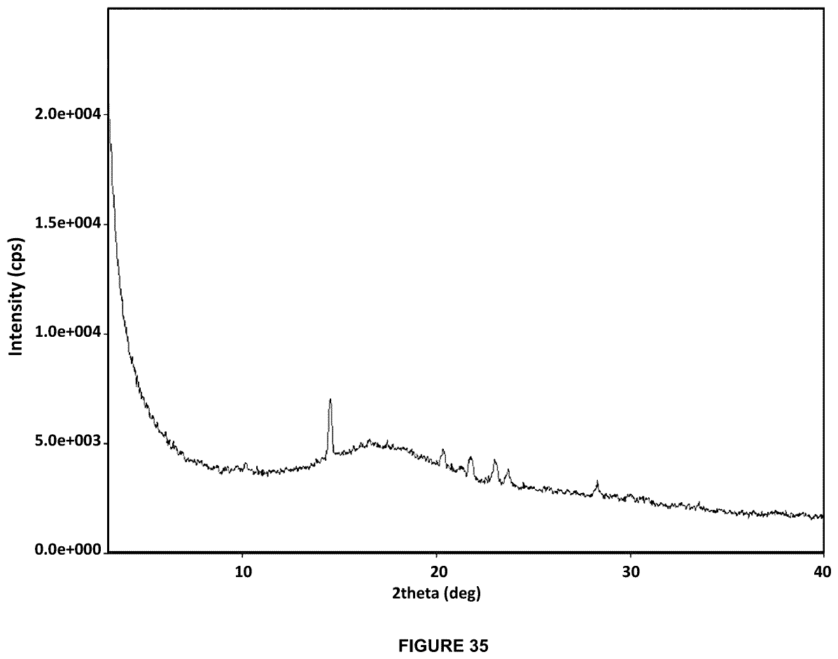

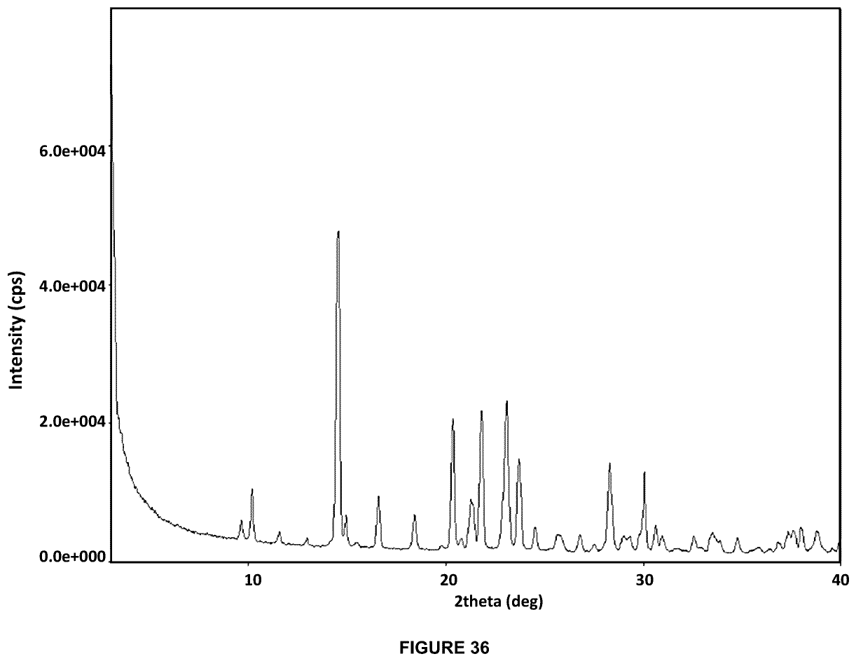

Solid 4-(3,3-difluoro-2,2-dimethylpropanoyl)-2,3,4,5-tetrahydropyrido[3,4-f][1,4]oxazepine-9-carbonitrile (˜100 mg) was added to a 2-dram vial and heated on a pie-block to >129° C. (melting point of Form A) resulting in a yellow oil. The vial was then flash cooled in a dry-ice/acetone bath to give a glassy yellow solid. DSC taken several hours later shows an exotherm at 87.8° C. followed by endotherm at peak 125.6° C. XRPD taken the following day appears mostly amorphous (see FIG. 35). This amorphous form was converted back to a crystalline form by heating at 90-100° C. on a pie-block (˜1-2 hr) and then allowing to cool to RT (see FIG. 36).

1-3. (canceled)

4. A crystalline solid form of 4-(3,3-difluoro-2,2-dimethyl-propanoyl)-3,5-dihydro-2H-pyrido[3,4-f][1,4]oxazepine-9-carbonitrile, wherein the crystalline solid form is at least 97% crystalline Form A and has an X-ray powder diffraction (XRPD) pattern derived using Cu (Kα) radiation comprising three, four, five, six, seven, or more peaks, in terms of 2-theta degrees, chosen from peaks at about 10.1±0.2, 14.3±0.2, 14.8±0.2, 16.4±0.2, 18.2±0.2, 20.1±0.2, 21.0±0.2,21.6±0.2, 22.8±0.2, 23.5±0.2, 28.1±0.2, 29.8±0.2.

5. The crystalline solid form of claim 4, having an XRPD pattern derived using Cu (Kα) radiation, in terms of 2-theta degrees, having peaks at about 14.3±0.2, 20.1±0.2, 21.6±0.2, 22.8±0.2, and 23.5±0.2.

6. The crystalline solid form of claim 4, having an X-ray powder diffraction pattern that is substantially in accordance with that shown in FIG. 1.

7. The crystalline solid form of claim 4, characterized by a differential scanning calorimetry (DSC) curve with an onset at about 128.5° C. and an endothermic peak at 129.6° C.

8. (canceled)

9. The crystalline solid form of claim 4, having an X-ray powder diffraction pattern that is substantially in accordance with any of those shown in FIG. 16, 17, 18, or 20.

10. A crystalline solid form of 4-(3,3-difluoro-2,2-dimethyl-propanoyl)-3,5-dihydro-2H-pyrido[3,4-f][1,4]oxazepine-9-carbonitrile, wherein the crystalline solid form is at least 97% Form A, and is characterized by at least two of:
a) an X-ray powder diffraction (XRPD) pattern substantially in accordance with that shown in FIG. 1;
b) an X-ray powder diffraction (XRPD) pattern derived using Cu (Kα) radiation comprising three, four, five, six, seven, or more peaks, in terms of 2-theta degrees, at about 10.1±0.2, 14.3±0.2, 14.8±0.2, 16.4±0.2, 18.2±0.2, 20.1±0.2, 21.0±0.2, 21.6±0.2, 22.8±0.2, 23.5±0.2, 28.1±0.2, 29.8±0.2;
c) a DSC/TGA profile substantially the same as shown in FIG. 2;
d) a Differential Scanning calorimetry (DSC) thermogram having an onset at about 128.5° C. and a peak at about 129.6° C.;
e) a TGA profile with an about 0.91% w/w loss from about 21.6° C. to about 120° C.;
f) an X-ray powder diffraction pattern that is substantially in accordance with that shown in FIG. 16;
g) an X-ray powder diffraction pattern that is substantially in accordance with that shown in FIG. 17;
h) an X-ray powder diffraction pattern that is substantially in accordance with that shown in FIG. 18; and
i) an X-ray powder diffraction pattern that is substantially in accordance with that shown in FIG. 20.

11-16. (canceled)

17. A process for preparing a crystalline polymorphic form of 4-(3,3-difluoro-2,2-dimethyl-propanoyl)-3,5-dihydro-2H-pyrido[3,4-f][1,4]oxazepine-9-carbonitrile having a purity of at least 97%, the process comprising the following steps:
a) dissolving 4-(3,3-difluoro-2,2-dimethyl-propanoyl)-3,5-dihydro-2H-pyrido[3,4-f][1,4]oxazepine-9-carbonitrile in a first solvent;
b) mixing the dissolved 4-(3,3-difluoro-2,2-dimethyl-propanoyl)-3,5-dihydro-2H-pyrido[3,4-f][1,4]oxazepine-9-carbonitrile with a second solvent to form a solution;
c) heating the solution;
d) cooling the solution to room temperature to provide a solid material;
e) filtering the solid material; and
f) drying the solid material to provide the crystalline polymorphic form,
wherein the second solvent is selected from at least one of methanol, acetone, ethyl acetate, cyclopentyl methyl ether, 2-methyltetrahydrofuran, acetonitrile, dichloromethane, 1,4-dioxane, dimethyl carbonate, tetrahydrofuran, and isopropyl alcohol.

18. The process of claim 17, wherein the crystalline polymorphic form has an X-ray powder diffraction (XRPD) pattern derived using Cu (Kα) radiation comprising three, four, five, six, seven, or more peaks, in terms of 2-theta degrees, chosen from peaks at about 10.1±0.2, 14.3±0.2, 14.8±0.2, 16.4±0.2, 18.2±0.2, 20.1±0.2, 21.0±0.2,21.6±0.2, 22.8±0.2, 23.5±0.2, 28.1±0.2, 29.8±0.2.
